(19) United States
(12) Patent Application Publication
(10) Pub. No.: US 2026/0283489 A1
(43) Pub. Date: Sep. 24, 2026

(54) STROKE DETECTION

(71) Applicant: Avertto Medical Ltd., Aderet (IL)

(72) Inventors: Hilla BEN-PAZI, Aderet (IL); Avshalom MIZRAHI, Zichron-Yaacov (IL); Limor Solange PRIGAN, RaAnana (IL); Lior TOLEDANO, Kiryat Ono (IL); Ori SHRIKI, Rehovot (IL); Pnina RAPPEL, Tel Aviv (IL); Meron Haim BEN-PAZI, Aderet (IL); Shirley ACKERMAN LAUFER, Kiryat Ono (IL); Yariv Avraham AMOS, Zurit (IL)

(73) Assignee: Avertto Medical Ltd., Aderet (IL)

(21) Appl. No.: 19/484,376

(22) PCT Filed: May 6, 2024
(86) PCT No.: PCT/IL2024/050430

§ 371 Date: Nov. 14, 2025

Related U.S. Application Data
(60) Provisional application No. 63/466,297, filed on May 14, 2023.

(51) Int. Cl. A61B 5/026; A61B 5/00; A61B 5/0205; A61B 5/024; A61B 5/318

(52) U.S. Cl. CPC A61B 5/026; A61B 5/0205; A61B 5/318; A61B 5/4064; A61B 5/6822; A61B 5/6867; A61B 5/746; A61B 5/7475; A61B 5/02438

(57) ABSTRACT

A method for determining changes in brain blood circulation, including:
recording signals from a single side of a neck;
processing the recorded signals;
determining hemodynamic changes in brain blood circulation indicating abnormal blood circulation at a contralateral side, based on results of the processing.

### FIELD AND BACKGROUND OF THE INVENTION

The present invention, in some embodiments thereof, relates to detection of abnormal blood flow and, more particularly, but not exclusively, to detection of detection of abnormal blood flow in carotid arteries.

Adequate blood supply to the brain is crucial to ensure regular brain activity. One medical condition that influences the adequate blood supply to the brain is brain stroke occurrence.

Brain stroke presents a possible abrupt deterioration in motor and/or cognitive function, the leading cause of disability. It is the second leading cause of death for people over 60. Over 15 million people in the world suffer from stroke a year. Six million people die annually from a stroke. Over 90% of survivors have a significant disability. Individual factors may also contribute to stroke risks. Age, diabetes, and hypertension increase the individual's odds further. With the aging population and expanding surgical services, these numbers will rise.

Stroke is usually classified as ischemic or hemorrhagic. The majority of strokes are ischemic. In ischemic stroke, upon diagnosis, intravenous and endovascular thrombolysis/therapies are viable options. Timely intervention cures ischemic stroke. It prevents irreversible neuronal death by effective revascularization. Treatment is only effective within the first hours. Every second, 2 million cells die. Thus, faster treatment leads to better outcomes. If left untreated, the patient suffers from a devastating outcome, lifelong disability, and/or death, whereas timely detection and treatment can improve quality of life.

Large vessel occlusions (LVO) are the most disabling. Recent studies have shown that endovascular thrombolysis is insufficient. Thrombectomy yields a much better outcome. Thus, the individual with LVO has to reach a stroke unit as fast as possible, preferably within an hour.

### SUMMARY OF THE INVENTION

Some examples of some embodiments of the invention are listed below (it should be noted that one or more features of an example may be used in combination with one or more features of another example):

Example 1. A method for determining changes in brain blood circulation, comprising:
recording signals from a single side of a neck;
processing the recorded signals;
determining hemodynamic changes in brain blood circulation indicating abnormal blood circulation at a contralateral side, based on results of said processing.

Example 2. A method according to example 1, wherein said determined hemodynamic changes indicate a pending ischemic event in the brain.

Example 3. A method according to example 2, comprising:
delivering an alert signal if said determined hemodynamic changes indicate said pending ischemic event.

Example 4. A method according to example 3, comprising detecting a location of said abnormal blood circulation and/or a location of a stenosis in a blood vessel, based on said determined hemodynamic changes, and wherein said delivered alert comprises information regarding said detected location.

Example 5. A method according to any one of examples 3 or 4, wherein said delivering comprises delivering said alert signal to said subject and/or to a remote device.

Example 6. A method according to any one of examples 3 or 4, wherein said alert signal includes information regarding initiation of a therapeutic time window and/or a duration of the therapeutic window.

Example 7. A method according to any one of the previous examples, comprising attaching at least one sensing unit to a skin surface of said neck at said single side, and wherein said recording comprises recording said signals by at least one detector of said at least one sensing unit.

Example 8. A method according to example 7, wherein said attaching comprises attaching said at least one sensing unit to a skin surface above a carotid artery in the neck.

Example 9. A method according to any one of examples 1 to 6, comprising implanting at least one detector at least partly into the neck or under the skin surface, at said single side, and wherein said recording comprise recording said signals by said at least one detector.

Example 10. A method according to example 9, wherein said implanting comprises implanting said at least one detector near a carotid artery.

Example 11. A method according to any one of examples 9 or 10, wherein said implanting comprises subcutaneously implanting said at least one detector.

Example 12. A method according to any one of the previous examples, comprising: measuring and at least one pulse wave based on said recorded signals, and wherein said identifying comprises identifying said hemodynamic changes based on said pulse wave measurements.

Example 13. A method according to example 12, wherein said measuring comprises measuring electrocardiogram (ECG), and wherein said identifying comprises identifying said hemodynamic changes based on said ECG and pulse wave measurements.

Example 14. A method according to example 13, wherein said recording comprises recording acoustic signals, and wherein said identifying comprises identifying said hemodynamic changes based on said acoustic signals and said ECG and pulse wave measurements.

Example 15. A method according to any one of examples 12 to 14, wherein said processing comprises extracting features which comprise morphological features and/or time based features from said at least one measured pulse wave, and wherein said determining comprises determining said hemodynamic changes based on said extracted features.

Example 16. A method according to example 15, wherein said processing comprises determining quality of said extracted features and/or in said at least one pulse wave, prior to said determining.

Example 17. A method according to any one of the previous examples, wherein said determining hemodynamic changes in brain blood circulation comprises determining hemodynamic changes in blood vessels delivering blood to the brain and/or blood vessels surrounding the brain, at said contralateral side.

Example 18. A method for determining changes in brain blood circulation, comprising:
implanting at least one detector at least partly in a neck tissue in a single side of the neck;
recording signals by said at least one detector;
processing said recorded signals
determining hemodynamic changes in brain blood circulation, based on results of said processing.

Example 19. A method according to example 18, wherein said determining hemodynamic changes in brain blood circulation comprises determining hemodynamic changes in blood vessels delivering blood to the brain and/or blood vessels surrounding the brain.

Example 20. A method according to any one of examples 18 or 19 comprising, implanting said at least one detector in a close vicinity to a carotid artery.

Example 21. A method according to any one of examples 18 to 20, wherein said implanting comprises subcutaneously implanting said at least one detector.

Example 22. A method according to any one of examples 18 to 19, wherein said implanting comprises implanting at least one additional detector at a different side of the neck.

Example 23. A method according to any one of the previous examples, comprising: measuring and at least one pulse wave based on said recorded signals, and wherein said determining comprises determining said hemodynamic changes based on said pulse wave measurements.

Example 24. A method according to example 23, wherein said measuring comprises measuring electrocardiogram (ECG), and wherein said determining comprises determining said hemodynamic changes based on said ECG and pulse wave measurements.

Example 25. A method according to example 24, wherein said recording comprises recording acoustic signals, and wherein said determining comprises determining said hemodynamic changes based on said acoustic signals and said ECG and said pulse wave measurements.

Example 26. A method according to any one of examples 23 to 25, wherein said processing comprises extracting features which comprise morphological features and/or time based features from said at least one measured pulse wave, and wherein said determining comprises determining said hemodynamic changes based on said extracted features.

Example 27. A method according to example 26, wherein said processing comprises determining quality of said extracted features and/or of said at least one pulse wave, prior to said determining said changes.

Example 28. A method according to any one of examples 18 to 27, comprising:
delivering an alert signal if said determining hemodynamic changes indicate abnormal blood circulation indicating a pending ischemic event in the brain.

Example 29. A method according to example 28, comprising detecting a location of said abnormal blood circulation and/or a location of a stenosis in a blood vessel, based on said determined hemodynamic changes, and wherein said delivered alert comprises information regarding said detected location.

Example 30. A method according to any one of examples 28 or 29, wherein said delivering comprises delivering said alert signal to said subject and/or to remote device.

Example 31. A method for detecting a pending ischemic event in a brain, comprising:
measuring signals from a location at a neck of a subject, wherein said signals comprise pulse waves signals;
processing said measured signals, wherein said processing comprises extracting features from said measured signals, and wherein said extracted features comprise morphological features and/or time based features;
detecting a pending ischemic event in the brain of said subject based on said extracted features and/or said measured signals.

Example 32. A method according to example 31, wherein said measured signals comprise electrocardiogram (ECG).

Example 33. A method according to any one of examples 31 or 32, wherein said measured signals comprise acoustic signals.

Example 34. A method according to any one of examples 32 or 33, wherein said extracted morphological features comprise at least one of, waveform amplitude, frequency, number of peaks, area under a curve, width, angle of slopes, timing, amplitude of peaks and/or shape.

Example 35. A method according to any one of examples 32 to 34, wherein said time based features comprise time intervals between specific points in a waveform signal.

Example 36. A method according to any one of examples 32 to 35, comprising: applying a model using said extracted features and/or said measured signals as input data for the model, and wherein said detecting said pending ischemic event comprises detecting said pending ischemic event based on an output of said model.

Example 37. A method according to example 36, comprising determining quality of said extracted morphological features and/or of said measured signals, prior to applying said model.

Example 38. A method according to any one of examples 31 to 37, comprising delivering an alert signal if a pending ischemic event is detected.

Example 39. A system for determining changes in brain blood circulation, comprising:
at least one sensing unit configured to be positioned at a side of a neck, comprising at least one pulse wave detector for measuring pulse waves;
a control unit functionally coupled to said at least one sensing unit, comprising:
a memory circuitry;
a control circuitry configured to receive said pulse wave measurement, to process said pulse wave measurements, and to determine hemodynamic changes in brain blood circulation indicating abnormal blood circulation at a contralateral side based on said processing results.

Example 40. A system according to example 39, wherein said control circuitry is configured to determine changes in said brain blood circulation indicating abnormal blood circulation at an ipsilateral side, based on said processing results.

Example 41. A system according to any one of examples 39 or 40, wherein said at least one sensing unit comprises at least one detector for measuring electrocardiogram (ECG), and wherein said control circuitry is configured to receive said ECG measurements and to process said ECG and pulse wave measurement.

Example 42. A system according to any one of examples 39 to 41, wherein said at least one sensing unit comprises at least one acoustic sensor configured to measure sound waves, and wherein said control circuitry is configured to receive said measured sound waves, and to determine said changes in said brain blood circulation based on said measured sound waves.

Example 43. A system according to any one of examples 39 to 42, wherein said at least one pulse wave detector comprises at least two waves detectors for measuring at least two pulse waves.

Example 44. A system according to any one of examples 39 to 43, wherein said at least one sensing unit is an implantable sensing unit comprising a flexible casing having an outer flat and smooth surface, wherein said flexible casing is shaped and sized to implanted into neck tissue.

Example 45. A system according to any one of examples 39 to 44, wherein said at least one sensing unit is a flexible skin patch configured to be attached to the skin surface via an adhesive layer on at least one surface of said skin patch.

Example 46. A system according to any one of examples 39 to 45, wherein said control circuitry is configured to process said pulse waves measurements by extracting morphological features and/or time based features from said pulse waves measurements, and to determine said changes in brain blood circulation based on said extracted morphological features and/or said extracted time based features.

Example 47. A system according to example 46, wherein said morphological features comprise at least one of, waveform amplitude, frequency, number of peaks, area under a curve, width, angle of slopes, timing, amplitude of peaks and/or shape.

Example 48. A system according to any one of examples 46 or 47, wherein said time based features comprise time intervals between specific points in a waveform signal.

Example 49. A system according to any one of examples 46 to 48, wherein said control circuitry is configured to provide said extracted morphological features and/or said extracted time based features as input data to a stroke detecting model stored in said memory circuitry, and to determine changes in brain blood circulation indicating said abnormal blood circulation which indicates a pending ischemic event, based on an output of said stroke detecting model.

Example 50. A system according to example 49, wherein said control circuitry is configured to provide said pulse waves measurements as input data to said stroke detecting model.

Example 51. A system according to any one of examples 49 or 50, wherein said control circuitry is configured to determine quality of said pulse waves measurements, said extracted morphological features and/or said extracted time based features, prior to using said stroke detecting model.

Example 52. A system according to any one of examples 49 to 51, wherein said control unit comprises a user interface configured to generate a human detectable indication, and wherein said control circuitry signals said user interface to generate said human detectable indication when changes in brain blood circulation indicating abnormal blood circulation are determined and/or when changes in brain blood circulation indicating a pending ischemic event are determined.

Example 53. A system according to any one of examples 49 to 52, wherein said control unit comprises a communication circuitry, and wherein said communication circuitry is configured to deliver an alert signal to a remote device when said changes in brain blood circulation are determined.

Example 54. A device for detecting an ischemic event in a subject, comprising:
a memory circuitry, wherein said memory circuitry stores pulse waves measurements;
a control circuitry, wherein said control circuitry is configured to extract features comprising morphological features and/or time based features, from said pulse waves measurements, and to identify changes in brain blood circulation indicating a pending ischemic event based on said extracted features.

Example 55. A device according to example 54, wherein said memory circuitry stores electrocardiogram (ECG) measurements, and wherein said control circuitry is configured to identify changes in said brain blood circulation indicating stroke pending ischemic event based on said extracted features and said ECG measurements.

Example 56. A device according to example 55, wherein said control circuitry is configured to select at least one set of pulse waves measurements from said stored pulse waves measurements using said ECG measurements, and to extract said features from said at least one selected set.

Example 57. A device according to any one of examples 54 to 56, wherein said morphological features comprise at least one of, waveform amplitude, frequency, number of peaks, area under a curve, width, angle of slopes, timing, amplitude of peaks and/or shape.

Example 58. A device according to any one of examples 54 to 57, wherein said time based features comprise time intervals between specific points in a waveform signal.

Example 59. A device according to any one of examples 54 to 58, wherein said memory stores a stroke detecting model, and wherein said control circuitry is configured to insert said extracted features and/or said pulse waves measurements as input data to said stroke detecting model, and to identify changes in brain blood circulation indicating stroke pending ischemic event based on an output of said stroke detecting model.

Example 60. A device according to example 59, wherein said memory stores acoustic signals, and wherein said control circuitry is configured to insert said acoustic signals as input data to said stroke detecting model.

Example 61. A device according to example 59 or 60, wherein said control circuitry is configured to determine quality of said extracted features and/or quality of said stored pulse waves measurements prior to providing input data to said stroke detecting model.

Example 62. A device according to any one of examples 54 to 61, comprising a communication circuitry, and wherein said communication circuitry signals said communication circuitry to deliver signals to a remote device with information regarding a detected pending ischemic event according to said identified changes in brain blood circulation indicating said pending ischemic event.

Example 63. A device according to any one of examples 54 to 61, comprising a user interface configured to generate a human detectable indication, and wherein said control circuitry signals said user interface to generate said human detectable indication with information regarding a detected pending ischemic event according to said identified changes in brain blood circulation indicating said pending ischemic event.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

As will be appreciated by one skilled in the art, some embodiments of the present invention may be embodied as a system, method or computer program product. Accordingly, some embodiments of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, some embodiments of the present invention may take the form of a computer program product embodied in one or more computer readable medium(s) having computer readable program code embodied thereon. Implementation of the method and/or system of some embodiments of the invention can involve performing and/or completing selected tasks manually, automatically, or a combination thereof. Moreover, according to actual instrumentation and equipment of some embodiments of the method and/or system of the invention, several selected tasks could be implemented by hardware, by software or by firmware and/or by a combination thereof, e.g., using an operating system.

For example, hardware for performing selected tasks according to some embodiments of the invention could be implemented as a chip or a circuit. As software, selected tasks according to some embodiments of the invention could be implemented as a plurality of software instructions being executed by a computer using any suitable operating system. In an exemplary embodiment of the invention, one or more tasks according to some exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized for some embodiments of the invention. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present invention may be described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a schematic illustration in a way of a block diagram, of a system for monitoring blood supply parameters to an individual's brain, according to some embodiments of the invention;

FIG. 2 is a schematic illustration in a way of a block diagram, of a system for monitoring blood supply parameters to an individual's brain utilizing baseline data, according to some embodiments of the invention;

FIG. 3 is a schematic illustration of a neck sensing unit configured for providing blood flow/velocity measurements, according to some embodiments of the invention;

FIG. 4 is a schematic illustration of a neck sensing unit including a two-dimensional sensor array, according to some embodiments of the invention;

FIG. 5 is a flow diagram of a process/method for determining a condition of blood supply to the brain, according to some embodiments of the invention;

FIG. 6A is a general flow chart of a process for identifying changes in blood flow to the brain and/or in the brain, and the detection of a stroke event, according to some embodiments of the invention;

FIG. 6B is a general flow chart of a process for identifying changes in blood flow to the brain and/or in the brain, and the detection of a stroke event, using at least one implanted detector, according to some embodiments of the invention;

FIG. 7 is a schematic illustration of showing a position of a sensing unit relative to arterial blood vessels in the neck and the brain of a subject, according to some embodiments of the invention;

FIGS. 8A and 8B are schematic illustrations showing a position of a sensing unit at a neck region, according to some embodiments of the invention;

FIGS. 9A-9C are block diagrams of a system for identifying changes in blood flow to and in the brain, and for detecting an ischemic event, according to some embodiments of the invention;

FIG. 9D is a schematic illustration showing a position of a sensing unit relative to a control unit, according to some embodiments of the invention;

FIGS. 10A and 10B are schematic illustrations of a device, which is placed in contact with the skin surface, according to some embodiments of the invention;

FIGS. 11A-11M are schematic illustrations showing different ways to apply a sensing unit or a detector to the skin surface or under the skin, according to some embodiments of the invention;

FIG. 11N is a schematic illustration showing communication between a sensing unit in the skin to a control unit on top of the skin, according to some embodiments of the invention;

FIG. 12A is a flow chart of general method for processing and analyzing measured signals for detecting an ischemic event, according to some embodiments of the invention;

FIG. 12B is a flow chart of a specific method for processing and analyzing measured signals for detecting an ischemic event, according to some embodiments of the invention;

FIG. 13 is a graph showing features of a waveform, according to some embodiments of the invention;

FIG. 14 shows examples of graphs of first six Orthonormal Hermite basis, according to some embodiments of the invention;

FIG. 15 shows examples of modeling of different waves with Hermite bases function, according to some embodiments of the invention;

FIGS. 16A and 16B are graphs showing a change in wave time between a normal aorta and a stiff aorta;

FIG. 17 is a flow chart showing processing of measured signals using a model, for example a deep learning model, according to some embodiments of the invention;

FIG. 18 shows changes in a peak-to-peak feature of a signal generated by data measured from an ipsilateral side, compared to a signal generated by data measured from a contralateral side; and

FIG. 19 is a flow chart of a process for communicating with a patient and/or with emergency services, when a pending ischemic event or a pending stroke is detected, according to some embodiments of the invention.

### DESCRIPTION OF SPECIFIC EMBODIMENTS OF THE INVENTION

The present invention, in some embodiments thereof, relates to detection of abnormal blood flow and, more particularly, but not exclusively, to detection of detection of abnormal blood flow in carotid arteries.

### Overview

An aspect of some embodiments of the invention relates to detecting or determining changes in blood circulation towards the brain and/or in the brain, indicating abnormal blood circulation at a specific side by measuring at least one hemodynamic parameter from a location at an opposite side of a subject neck. In some embodiments, the detected changes in blood circulation indicate an ischemic event, for example a stroke event in the brain. As used here, detection of an ischemic event or a stroke event includes also detection of a pending ischemic event. In some embodiments, the measurements of the at least one hemodynamic parameter are performed from a location in a vicinity to at least one carotid artery. In some embodiments, the measured changes allow to detect changes in blood circulation at both ipsilateral and contralateral sides of the neck and/or brain, which may indicate a brain ischemic event in the subject.

According to some embodiments, the determined changes in blood circulation comprises hemodynamic changes, which indicate an abnormal blood circulation towards the brain and/or in the brain. In some embodiments, detecting of a pending ischemic event means detecting of an ischemic event prior to formation of brain damage, prior to a formation of irreversible damage to the brain and/or prior to cell death in the brain, due to abnormal blood circulation. In some embodiments, the determined hemodynamic changes which indicate abnormal blood circulation may be a result of changes in at least one brain arterial blood vessels, at least one brain venous blood vessels, dilation of at least one blood vessel delivering blood to the brain or in the brain, constriction of at least one blood vessel delivering blood to the brain or in the brain, and/or partial or complete blockage of at least one blood vessel delivering blood to the brain, or in the brain.

According to some embodiments, the hemodynamic changes are detected by at least one detector, for example a sensor, positioned at the single location of the neck. In some embodiments, the at least one detector is positioned on a skin surface of the neck. Alternatively, the at least one detector is implanted at least partly or completely within the neck. In some embodiments, the at least one detector comprises at least one of, an electrocardiogram (ECG) detector, a pulse wave detector and/or an acoustic signals detector. In some embodiments, the at least one detector comprises two or more detectors located in at least one, optionally single, sensing unit. In some embodiments, the sensing unit is positioned on top of the skin surface of the neck, optionally attached to the skin surface of the neck. Alternatively, the sensing unit is implanted at least partly or entirely within the neck tissue.

According to some embodiments, the at least one detector or the sensing unit is in communication with a control unit, via wireless communication and/or by wired communication. In some embodiments, the control unit received signals from the at least one detector or sensing unit, processes the signals and/or transmits the received or processed signals to a remote device. In some embodiments, the control unit is an implanted control unit. Alternatively, the control unit is located outside the subject body, and is optionally positioned on top of the skin surface. Optionally, the control unit is attached to the skin surface.

According to some embodiments, at least one signal measured by the at least one sensing unit and optionally in combination with other signals measured by one or more detectors located on the subject body, are used to detect an ischemic event, for example a stroke event, in the subject. In some embodiments, a control unit and/or a remote device in communication with the control unit, detects that a stroke event has initiated or is in progress in the subject, in either side of the brain, based on the measured signals. In some embodiments, the control unit and/or the remote device estimate or identify a location of the ischemic event based on the measured signals. In some embodiments, identifying an ischemic event location comprises determining an axial distance of the ischemic event location from the location in which the at least one sensor or the sensing unit is positioned.

According to some embodiments, the changes in brain blood circulation, for example hemodynamic changes, are identified based on pulse waves measurement, for example at least one channel of pulse waves measurements or at least two channels of pulse waves measurements. In some embodiments, electrocardiogram (ECG) measurements are also used with the pulse waves measurements to identify the changes in brain blood circulation. In some embodiments, the ECG measurements are used to select at least one set of pulse waves measurements for further analysis and/or processing.

An aspect of some embodiments relates to implanting at least one sensing unit in a neck of a subject for detecting changes in blood circulation towards the brain and/or in the brain. In some embodiments, the detected changes indicate a pending ischemic event, for example a pending stroke event. In some embodiments, the at least one sensing unit is used to measure pulse waves, for example at least one channel or at least two channels of pulse waves. Additionally or optionally, the sensing unit is configured to measure electrocardiogram (ECG). In some embodiments, the at least one sensing unit is implanted in vicinity to a common carotid artery or derived arterial blood vessels. In some embodiments, at least two sensing units are implanted, each at a different side of the neck.

According to some embodiments, the implanted at least one sensing unit is functionally coupled to an implanted control unit, optionally implanted in a chest of the subject. Alternatively the implanted at least one sensing unit is functionally coupled to a control unit positioned outside of the subject body. In some embodiments, the control unit is attached to the skin surface above the implanted sensing unit. In some embodiments, the implanted sensing unit is in wireless communication with the control unit.

An aspect of some embodiments relates to detecting changes in blood circulation in the brain and/or towards the brain based on pulse waves measurements, optionally in combination with ECG measurements. In some embodiments, the measurements are performed from one or more locations at a neck of a subject. In some embodiments, morphological features and/or time based features extracted from the ECG measurements and/or from the pulse wave measurements are used for detecting the changes in brain blood circulation, indicating abnormal blood circulation. In some embodiments, the extracted features are used as input for a model, for example a stroke detecting model. In some embodiments, the stroke detection model, is a deep learning model, that generates an output score indicating a probability of having an ischemic event in the subject brain. In some embodiments, the model is a classification model. As used herein, a stroke detection model is a model that detects a pending ischemic event, for example a pending stroke event, and provides an output signal if a pending ischemic event is detected. In some embodiments, the model allows to provide early warning to a subject or to a health care provider, or to a telemedicine service, that the subject is in a pending ischemic event state.

According to some embodiments, the morphological features comprise at least one of, waveform amplitude, frequency, number of peaks, area under a curve, width, angle of slopes, timing, amplitude of peaks and/or shape. In some embodiments, the time based features comprise time intervals between specific points in a waveform signal.

According to some embodiments, the system determines a quality of the measurements and/or the extracted features, for example using an artifact detecting model, for example a traffic light artifact detection model. In some embodiments, the system determines the quality using the model, before using the measurements and/or the extracted features as input data for the stroke detecting model.

According to some embodiments, the classification model is personalized for a specific subject, a specific group of subject, a specific clinical state of the subject or group of subjects. In some embodiments, the output score generated by the classification model is measured over time, and is optionally stored.

According to some embodiments, a quality detection model is applied on the pulse wave measurements, for determining a quality of the pulse wave signals prior to insertion of the pulse waves extracted features to the classification model. In some embodiments, the quality detection model is based on Convolutional Neural Network (CNN).

According to some embodiments, the systems and/or methods described herein provide a novel approach for monitoring blood supply to the brain. In some embodiments, the present invention provides systems, devices, and methods for alerting about the possibility of imminent brain stroke or the insufficient blood supply to the brain, which affects brain perfusion, thereby enabling early intervention to keep quality of life or save lives. Large populations are at risk for stroke, individuals over 65, and/or with comorbidities such as diabetes and hypertension. Individual factors may also contribute to stroke risks. With increased longevity, the risk lasts years. Thus, an effective monitoring system must be permanently attached to optionally override compliance challenges.

According to some embodiments, the novel systems in the present invention are robust yet feasible and cost-effective, specifically designed for continuous monitoring over the years for populations at risk. In some embodiments, the invention will alert about the possibility of stroke, optionally enabling timely diagnosis and intervention. In some embodiments, the invention is based, inter alia, on detecting hemodynamic changes indicating a stroke. In some embodiments, the invention can identify hemodynamic features/changes that indicate a stroke or a pathologic change in blood supply to the brain.

According to some aspects of the invention, analysis of one or more hemodynamic parameters measured directly on the user's body by one or more sensors or indirectly obtained from the measurements by the sensor(s) provides an indication about the blood supply to the brain, e.g., indication about stroke occurrence or insufficient perfusion. In some embodiments, the sensor(s) is (are) located near the carotid artery upon or in the body. In some embodiments, the measurements (indicative of hemodynamic parameters) are obtained from the carotid artery(ies) located on the neck's side(s). In some embodiments, the measurements are obtained from the common and external carotid arteries located on one side of the neck. In some embodiments, the measurements are obtained from the internal and external carotid arteries located on one side of the neck. In some embodiments, the measurements are obtained from the two common carotid arteries located on both sides of the neck.

According to some embodiments, a neck sensing unit, including at least one sensor, may be used for the measurements near the monitored blood vessel. For example, at least one sensor may be comfortably inserted over and/or under the skin on one or both sides of the neck to monitor carotid arteries. In some embodiments, the sensor(s) utilize(s) one or more modalities, individually or collectively, sequentially or simultaneously, to provide the measurements of the hemodynamic parameters, such as optical (PPG) pressure based, and electrical (e.g., capacitive, ECG) measurement modalities. In some embodiments, the analysis of the measurements points out an occurring/imminent stroke or another condition of the change in blood supply to the brain.

In some embodiments, the analysis compares the measured hemodynamic parameter(s) to corresponding hemodynamic history data. In some embodiments, the hemodynamic history data may include baseline data possibly obtained prior to collecting the data indicative of the occurring/imminent stroke. In some embodiments, the baseline data may include personal baseline data obtained from the same monitored individual or collective baseline data obtained from a plurality of previously monitored individuals and saved in an accessible database.

In some embodiments, Artificial Intelligence and/or Machine Learning may be applied to the hemodynamic history/baseline data accumulated over a predetermined time period from the individual(s) using the principles of the present invention to generate dynamic reference hemodynamic data that forms at least part of the hemodynamic history data to which the personal measured data is compared.

According to some embodiments, the hemodynamic history data may include other quantitative data that indicate a possibility of a stroke, e.g., quantitative data that has been recognized by the medical field as indicating a stroke occurrence, possibly based on medical research (for example, a unilateral decrease of over 20% for more than 30 seconds).

In some embodiments, the analysis relates to hemodynamic data measured/obtained from at least one sensor. In some embodiments, in the case of one sensor, the hemodynamic data is collected from a specific blood vessel. Alternatively, two or more sensors are used. In some embodiments, the two measurements are extracted from the same side of the neck. In some embodiments, the measured data obtained from a blood vessel is compared to the previous data of that blood vessel. In some embodiments, measurements from a single side of the neck is used to generate an indication regarding blood flow via arteries in both sides of the neck.

According to some embodiments, a system for monitoring of blood supply to a brain of an individual, the system comprising:
A neck sensing unit configured and operable to be placed over and/or under the skin near the carotid artery and collect over time, from at least one blood vessel, measured data indicative of one or more hemodynamic parameters; and
A control and processing unit in communication with the sensing units, the control and processing unit is configured and operable to receive and analyze said measured data, determine hemodynamic data comprising said one or more hemodynamic parameters, analyze said hemodynamic data, and upon detecting a predetermined change over time, generate output data indicative of a blood supply condition to the brain of the individual. In some embodiments of the invention, the monitoring system detects local changes in blood flow to the brain, optionally correlated with various physiological parameters to provide stroke alerts.

In some embodiments, hemodynamic changes are detected with a local system that includes at least two sensors, with at least one sensor for each carotid artery on the right and/or left sides, and a controller designed to control process, analyze and send alerts. In some embodiments, stroke detection involves acute arterial hemodynamic changes compared to a baseline, especially the variation between the sensors.

In some embodiments, the control and processing unit is configured and operable to analyze the first portion of said measured data collected over the first time period, determine a corresponding first portion of the hemodynamic data and save the first portion of the hemodynamic data as a baseline data, analyze the second portion of said measured data continuously collected over the second time period and determine a corresponding second portion of the hemodynamic data, apply a comparison between the second portion of the hemodynamic data and a history data comprising the baseline data, to generate said output data indicative of a blood supply condition to the brain of the individual. The history data may comprise baseline and/or event data of one or more monitored individuals. The event data can be, for example, historical data relaying the expected changes in hemodynamic parameters indicating the occurrence of stroke.

In some embodiments, the control and processing unit is configured and operable to analyze systemic hemodynamic changes from baseline and apply a comparison between the hemodynamic history data and the newly acquired hemodynamic data to generate said output data indicative of a blood supply condition to the brain of the individual. In some embodiments, the control and processing unit is wirelessly connected to the said sensing units. In some embodiments, the sensing units and said control and processing unit are located on a common platform.

In some embodiments, the neck sensing unit comprises an array of sensors configured and operable to collect the measured data from the carotid artery along with a predetermined distance thereof. In some embodiments, the sensing unit comprises a two-dimensional array of sensors configured to be placed in a vicinity of an area of the individual's neck covering said carotid artery; said control and processing unit is configured and operable to activate one or more sensors of the two-dimensional array of sensors to collect the measured data.

In some embodiments, the neck sensing unit comprised a capacitive sensor, and said hemodynamic data comprises one or more of the following: blood vessel expansion data and blood flow data.

In some embodiments, the neck sensing unit comprises a sensor (pulse, ECG temperature, movement) configured and operable to collect the measured data over time in a reliable manner.

In some embodiments, the sensing unit comprises at least one of, a piezoelectric sensor, a magnetic sensor, an accelerometer, and/or a fiberoptic.

In some embodiments, the sensing unit comprises a pressure (barro) sensor.

In some embodiments, the sensing unit comprises a PPG sensor.

In some embodiments, the sensing unit comprises a resistive/optical strain gauge sensor.

In some embodiments, where the neck sensor unit is optically based, whether a single sensor, array of sensors, or imaging sensor, the light can be transmitted and/or received via optical fibers.

In some embodiments, at least one blood vessel is one or more of the following: common carotid artery, external carotid artery, and internal carotid artery.

In some embodiments, the neck sensing unit comprises a temperature sensor.

In some embodiments, the bed sensing unit comprises a piezoelectric sensor.

In some embodiments, the bed sensing unit comprises a pressure sensor.

In some embodiments, the control and processing unit is configured and operable to generate the output data being indicative of an increase or a decrease of blood supply to the brain.

In some embodiments, the control and processing unit is configured and operable to generate the output data being indicative of a stroke occurring in the individual's brain.

In some embodiments, the control and processing unit is configured and operable to generate the output data being indicative of an increase or a decrease of hemodynamic parameters (such as heart rate variability), indicative of a stroke.

In some embodiments, the control and processing unit is configured and operable to receive and analyze medical data in addition to the hemodynamic data to generate thereby the output data indicative of a blood supply condition to the brain of the individual.

In some embodiments, the medical data comprises one or more of the following: EEG data, ECG data, pulse data, Emboli data, and/or carotid artery hemodynamic data.

According to some embodiments, there is provided a method for determining a blood supply to brain condition, the method comprising: receiving measured data collected over time from at least one blood vessel; analyzing the measured data and determining hemodynamic data comprising one or more hemodynamic parameters; analyzing the hemodynamic data and upon detecting a predetermined change in one or more hemodynamic parameters determining the condition of the blood supply to the brain.

In some embodiments, the measured data comprises first and second measured data collected respectively over first and second periods, said analyzing of the measured data comprising determining first and second hemodynamic data respectively, and said analyzing of the hemodynamic data comprising comparing the second hemodynamic data with the first hemodynamic data for detecting the predetermined change in the one or more hemodynamic parameters and determining the condition of blood supply to the brain. In some embodiments, the first hemodynamic data forms baseline data saved and used for comparing with hemodynamic data collected later on.

In some embodiments, analyzing the hemodynamic data comprises comparing the hemodynamic data to baseline and/or event data comprising hemodynamic data of one or more individuals who have been monitored.

In some embodiments, the measured data is collected from at least two blood vessels located respectively on the right and left sides of a neck of the individual, the measured data thereby comprising right and left measured data respectively, and the hemodynamic data comprising right and left hemodynamic data respectively.

In some embodiments, the analysis of the hemodynamic data comprises analyzing each of the right and left hemodynamic data and applying a comparison between the respective hemodynamic data and a respective baseline data determined based on the first portion of the respective hemodynamic data to determine the blood supply condition to the brain of the individual.

In some embodiments, the analysis of the hemodynamic data comprises applying a comparison between the right and left hemodynamic data to determine the blood supply condition to the individual's brain.

In some embodiments, the hemodynamic data comprises one or more of the following: blood vessel's expansion data and blood flow data.

In some embodiments, at least one blood vessel is one or more of the following: common carotid artery, external carotid artery, internal carotid artery, and jugular vein.

In some embodiments, the blood supply to brain condition is indicative of a stroke occurring in the individual's brain.

In some embodiments, the blood supply to brain condition is indicative of an increase or a decrease of blood supply to the brain.

In some embodiments, the method comprises receiving and analyzing medical data in addition to the hemodynamic data for determining the blood supply condition to the brain of the individual. The medical data may comprise one or more of the following: EEG data, Emboli data, ECG data, Heart Rate data, and carotid artery and/or jugular vein hemodynamic data.

As used here, detection of an ischemic event or a stroke event may include also detection of a pending ischemic event. According to some embodiments, the determined changes in blood circulation comprises hemodynamic changes, which indicate an abnormal blood circulation towards the brain and/or in the brain. In some embodiments, detecting of a pending ischemic event means detecting of an ischemic event prior to formation of brain damage, prior to a formation of irreversible damage to the brain and/or prior to cell death in the brain due to the determined hemodynamic changes. In some embodiments, the determined hemodynamic changes which indicate abnormal blood circulation may be a result of changes in at least one brain arterial blood vessels, at least one brain venous blood vessels, dilation of at least one blood vessel delivering blood to the brain or in the brain, constriction of at least one blood vessel delivering blood to the brain or in the brain, and/or partial or complete blockage of at least one blood vessel delivering blood to the brain, or in the brain.

According to some embodiments, once hemodynamic changes which indicate abnormal blood circulation in the brain are detected, an alert signal is generated and is optionally delivered to at least one of, a subject, a caregiver, a health care professional, a telemedicine service monitoring the subject condition. In some embodiments, the alert signal includes information on a pending ischemic event, indicated by the abnormal blood circulation and/or the determined hemodynamic changes. In some embodiments, the alert signal provides information regarding a therapeutic time window, which allows, for example, to treat the subject prior to damage formation or formation of irreversible damage, to brain tissue, which may lead to paralysis of the subject. In some embodiments, the alert signal provides information regarding the location of a source of the abnormal blood circulation, the distance of the source from at least one measurement site in the neck, time in which the therapeutic window has initiated, duration of the therapeutic window, time remaining to the closure of the therapeutic window, and/or time in which the therapeutic window closes.

According to some embodiments, the system, device and/or methods described herein, are used during a surgical procedure, for example in an operating room. In some embodiments, the detected hemodynamic changes are used to provide an alert indication for developing an ischemic event, for example a pending ischemic event during surgery.

According to some exemplary embodiments, the system, device and/or methods described herein, are used to monitor a state of a subject, for example a patient, when the subject is asleep, for example at home. Alternatively or additionally, the system, device and/or methods described herein are used to monitor a state of a subject, when the subject is sedated, optionally undergoing a surgical procedure.

Potential advantages of having the system and/or device described herein that continuously monitor hemodynamic flow of a subject, may be to prevent disability of a subject by enabling early detection of a pending ischemic event, for example, a pending stroke event, and intervention in case of a stroke event, prior to formation of damage or formation of irreversible damage.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary System for Monitoring Brain's Blood Supply Conditions/Parameters

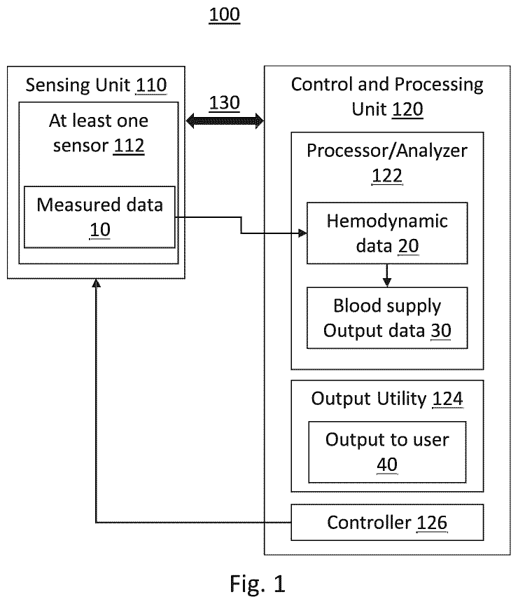

Reference is made to FIG. 1 that illustrates a non-limiting example of a system 100 for monitoring the brain's blood supply conditions/parameters, according to some exemplary embodiments of the invention.

According to some exemplary embodiments, system 100 includes a neck sensing unit 110 and a control and processing unit 120 configured to communicate via a communication assembly/network/protocol 130.

In some embodiments, the neck sensing unit 110 includes at least one sensor 112 configured and operable to collect measured data 10 indicative of one or more hemodynamic parameters of the monitored individual.

In some embodiments, the neck sensing unit 110 is configured and operable to be positioned in the vicinity of at least one blood vessel carrying blood to/from the brain. For example, the neck sensing unit 110 may be attached to the individual's neck to monitor hemodynamic parameter(s) from the carotid artery(ies). Ascending carotid arteries carrying blood to the brain are located on the right and left sides of the neck.

For example, at least one sensor 112 may be attached to the individual's neck in the vicinity of one or more of the following: the common carotid artery, the internal carotid artery (stemming from the common carotid artery and supplying blood to the brain arteries), and/or the external carotid artery (stemming from the common carotid artery and supplying blood to the facial area).

As mentioned above, in some embodiments, the neck sensing unit 110 may include one or more sensors 112, each of the sensors is configured and operable to collect data in one or more measurement modalities (e.g., optical, ultrasound, capacitive). Some non-limiting examples of the sensor(s) 112 are described further below.

According to some exemplary embodiments, the control and processing unit 120 includes a processor/analyzer 122 configured and operable to receive, process, and analyze the measured data 10 and generate output hemodynamic data 20 and output data 30 indicative of the brain blood supply condition, and an output utility 124 configured and operable to generate an output to a user 40 indicative of the blood supply condition, such as an alert to the user. In some embodiments, the control and processing unit 120 includes a controller 126 configured and operable to control the neck sensing unit 110 and collect the measured data 10. In some embodiments, the controller 126 is also configured and operable for providing Quality Assurance that the signal detected by the neck sensing unit 110 is stable and reliable. In some embodiments, the controller can alert if the person is in bed but without hemodynamic sensing/the controller can detect hemodynamic sensing when the person is not in bed.

In some embodiments, system 100 is enclosed in a single housing accommodating both the sensing units 110, and the control and processing unit 120. In some embodiments, the neck sensing unit 110 and the control and processing unit 120 are accommodated in two different housings. In some embodiments, part of the control and processing unit 120 is located within the same housing as the sensing unit 110 (such as the controller 126), while another part is located in another housing. In some embodiments, the control, and processing unit 120 is distributed between at least two housings/locations (including remote locations such as a remote server/cloud server). In some embodiments, the control, and processing unit 120 is at least partially implemented as a software module in a computing device.

In some embodiments, the control and processing unit 120 communicates with the sensing unit 110 via the communication assembly/network/protocol 130, utilizing a communication technique known in the field, either wired or wireless communication, such as Bluetooth or Wi-Fi communication. Optionally, the system 100 and/or the communication assembly 130, include (not explicitly illustrated in the figures):
A/D converter (may be integral with the sensing units 110), depending on the transmission protocol for transmitting the signals detected by the sensing unit 110 to the control and processing unit 120. Optionally, the A/D converter should be of at least 8 bits to meet the specific application requirements.
Bluetooth/Wi-Fi/other transmitters for transmitting the signals to the control and processing unit 120 or a wiring network. In some embodiments, the method of transmitting the data, whether wired or wireless, depends on safety, environmental and ergonomic considerations;
A power supply, for example a battery may be included for powering the control unit, the communication assembly and/or the sensing units. In some embodiments, the battery should preferably be small, lightweight, and flat, designed to allow for continuous operation of enough time, for example, at least a few hours; a battery may be included in the control unit and/or in processing unit;
A receiver (that may form part of the control and processing unit 120), designed to collect signals from the sensing unit 110, allowing for possible synchronous operation when a plurality of sensors are present. In some embodiments, where the monitored hemodynamic parameter is the pulse wave of the blood, the receiver is adapted to a sampling rate of more than 10 Hz.

In some embodiments, the processor/analyzer 122 processes/analyzes the measured data 10 received from the sensing unit 110 to determine a hemodynamic parameter 20 such as blood velocity, blood flow, blood volume, heart rate variability, vessel volume, blood characteristics such as oxygen or other constituents. Optionally, the processor/analyzer 122 then analyzes the hemodynamic parameter(s) 20. In some embodiments, upon detecting a predetermined change over time in the hemodynamic parameter(s), individually or among different parameters, the processor/analyzer 122 generates output data 30 indicative of the blood supply to the brain. Optionally, based on the output data 30, the output utility 124 generates a corresponding output to the user, e.g., a perfusion alert or a stroke alert. In some embodiments, the processor/analyzer 122 generates the output data 30 once there is a difference above a predetermined threshold between the measured data 10 or the processed hemodynamic data 20 and baseline data, as will be further described below.

According to some exemplary embodiments, systems and methods for monitoring blood supply to the brain of an individual are presented herein. In some embodiments, the system comprising a neck sensing unit configured to be placed in a vicinity of at least one blood vessel and operable to collect over time, from the at least one blood vessel, measured data indicative of one or more hemodynamic parameters.

In some embodiments, the system optionally comprises bed sensors measuring pressure and/or movements. In some embodiments, the system comprises a control and processing unit in communication with the sensing units.

According to some exemplary embodiments, the control and processing unit being operable to receive and analyze the measured data, determine hemodynamic data comprising the one or more hemodynamic parameters, analyze the hemodynamic data with movement data and, upon detecting a predetermined change over time in the hemodynamic data, generate output data indicative of a blood supply condition to the brain of the individual.

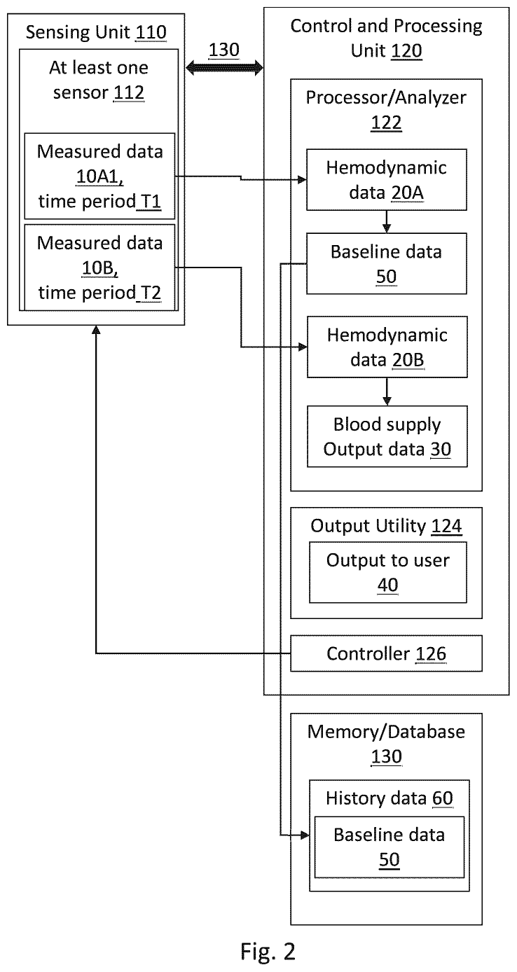

Exemplary analysis of measured data Reference is made to FIG. 2, illustrating a non-limiting example of the analysis of measured data by the control and processing unit 120 for determining the blood supply condition data 30, according to some exemplary embodiments of the invention.

According to some exemplary embodiments, the control and processing unit 120 is configured and operable to determine the output data 30 by analyzing a first portion of the measured data 10A1 collected over the first period T1, determining a corresponding first portion of the hemodynamic data 20A and saving the first portion of the hemodynamic data 20A as a baseline data 50, possibly in a local/distant memory or database 130. In some embodiments, the control and processing unit 120 then analyzes a second portion of the measured data 10B continuously collected over the second period T2 and determines a corresponding second portion of the hemodynamic data 20B. In some embodiments, the control and processing unit 120 then optionally applies a comparison between the second portion of the hemodynamic data 20B and history data 60 that includes the baseline data 50 and possibly other data as will be further described below and generates the output data 30 indicative of the blood supply condition to the brain of the individual.

Optionally, the baseline line data 50 can be dynamic data that is continuously updated. So, for example, the baseline data can be updated with the hemodynamic data 20B. Further, in some embodiments, the baseline data 50 and/or generally the history data 60 can be based on measured data from the same individual or measured data from a plurality of individuals.

As used herein, as appreciated, the baseline data 50 typically refers to the characteristic/value of the same hemodynamic parameter in the same blood vessel during a presumed normal/healthy period. Alternatively, the baseline data 50 of the hemodynamic parameter may refer to a relation (e.g., difference, division, etc.) or correlation between the values of the hemodynamic parameter in different blood vessels, such as in carotid blood vessels. Nevertheless, the baseline data 50 may refer to a relation or correlation between two or more hemodynamic parameters measured in the same blood vessel or different blood vessels. In some embodiments, the baseline data 50 may be calculated as a mean value and/or as an average of the hemodynamic parameter's characteristic (e.g., value) monitored during a period prior to the stroke/blood supply condition. In some embodiments, for example, as described above, the baseline data 50 may be obtained from the same individual during a predetermined period, and as such forms, personal baseline data, or the baseline data may be collective baseline data obtained from a plurality of individuals and updated with each newly monitored individual including the currently monitored individual. It is appreciated that the history data 60, to which the hemodynamic parameters 20 of the currently monitored individual are compared, may optionally include both the personal and the collective baseline data. Also, it is appreciated that the history data 60 may optionally include hemodynamic data that the medical field has recognized (e.g., through research, applying machine learning algorithms, etc.) as being indicative of a stroke or a condition of the blood supply to the brain.

In some embodiments, the monitored hemodynamic data 20 comprises a parameter of blood propagation (e.g., pulse wave velocity) in the carotid arteries. The main anatomical definitions related to brain stroke location are Ipsilateral—the same side as the stroke and Contralateral—the opposite side of the stroke. If the stroke occurs on the left side of the brain, the left side is the ipsilateral side, and the right side is the contralateral side. At baseline, e.g., in a normal/healthy situation, blood flow towards the brain and/or from the brain is more or less constant. A difference, if exist between flow in the right side of the neck and brain to flow in the left side of neck and brain, remains constant. For example, a difference between the flow in the right common carotid (CC) and the flow in the left common carotid, and vice versa, remains constant in normal/healthy conditions. Usually at each side of the neck, most of the blood flows through the larger internal carotid (IC) and less through the external carotid (EC).

As an ischemic event, for example a stroke, evolves, there is a change in blood flow. According to some exemplary embodiments, the system, device and/or method described here, are configured to detect the change in blood flow that optionally indicates an ischemic event in the brain. In some embodiments, the system, device and/or method described here, are configured to detect the change in blood flow by measuring signals from a single side of the body, for example a single side of the neck.

When a stroke occurs, there will be, compared to the baseline, hemodynamic changes that could be detected. Accordingly, in some embodiments of the invention, initial baseline data (e.g., blood flow in a normal/healthy/pre-stroke condition) is obtained for each sole patient and used for comparison with the measured data collected thereafter during a stroke. Optionally, the baseline is derived from a group or a population of subject, for example at least one of, subjects that have similar medical history, subjects within a similar age range, in a similar age group, subjects having the same gender, subject that have a similar family and/or social background.

### Exemplary Hemodynamic Sensor/Detector

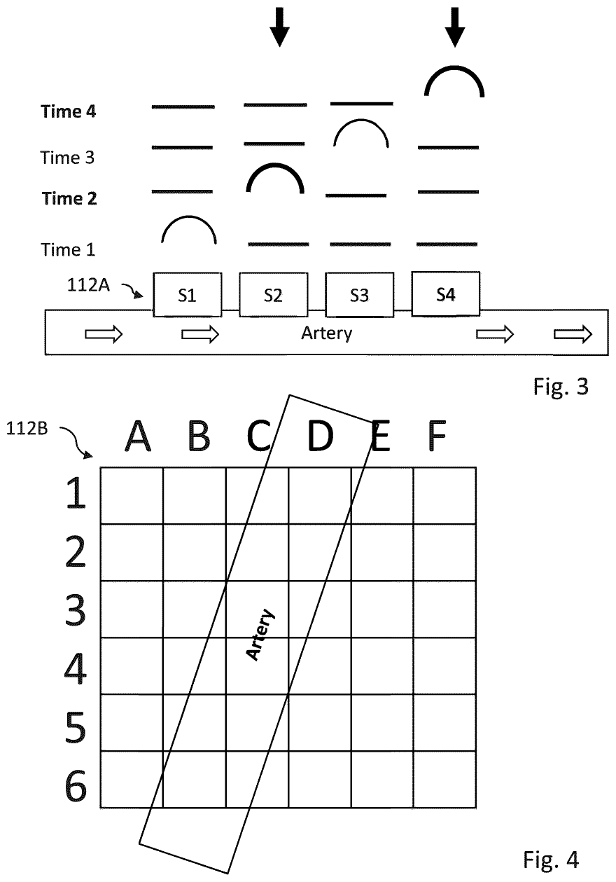

Reference is now made to FIG. 3 schematically illustrating a non-limiting example of at least one sensor, which is a hemodynamic sensor/detector, according to some exemplary embodiments of the invention.

According to some exemplary embodiments, at least one detector, for example a sensor is used. In some embodiments, a sensor array 112A of four sensors S1-S4 (an array of at least two sensors is required) is shown lying along an axis of an artery. In some embodiments, each sensor can record a change over time in at least one parameter related to blood pulse in the artery portion beneath the sensor. In a non-limiting example, in some embodiments, such sensors can be capacitance-based sensors detecting the pulsation of the blood flowing in the artery by the change of the artery's diameter as reflected by a change in the skin displacement/movement. Optionally, the chosen sampling rate enables to capture of the change in the pulse waveform over time, represented as time 1-time 4, such that sensor S1 records the pulse waveform at time 1, sensor S2 records the pulse waveform at time 2, and so on. In some embodiments, each sensor along the way can detect the wave of pulsation of the blood, such that the location of the sensor along the artery and the distances between the sensors can indicate the velocity of the pulse propagation (pulse wave velocity PWV). The PWV is the distance passed over time. In the described example, it can be said that:
PWV=distance/time=distance between any two sensors/time difference of recording the propagating pulse.

Measuring the pulse/waveform characteristics, such as velocity, using an array or a matrix of sensors could be achieved in some embodiments by other sensor types, such as ultrasound sensors detecting the echo (Doppler) from the artery or optical sensors detecting changes in absorption of light as a function of time, or other types as listed further below.

According to some exemplary embodiments, the control and processing unit is operable to analyze the pulse/waveform characteristics, including the shape, area, maximum, minimum, width, and others.

In some embodiments, in one non-limiting example, the sensor(s) can be made out of piezoelectric material and operable, when attached to the body surface above the blood vessel of interest, to generate electrical signals as a function of mechanical variations occurring below the sensor(s) as a result of the blood flow. In one non-limiting example, the sensor(s) is (are) made from at least one of the following: Bimorph Ceramic material or Polyvinylidene Fluoride (PVDF).

In some embodiments, each sensor in the sensor array can be separate, or the sensor array can be mounted on a common mechanical support. The latter option is easier for attaching the sensor array to the body. As appreciated, in some embodiments, any sensor unit is flexible to conform to the body's geometry (e.g., the neck).

In some embodiments, the electrical circuit that transmits the electrical signal from the sensing units to the control and processing unit can be attached directly to the mechanical support of the sensing units or placed at a distance from the sensor(s) to decrease the size and weight of the sensing units.

Optionally, in one non-limiting example, the sensing units include resistive/optical strain gauge sensor(s).

In yet another non-limiting example, in some embodiments, the sensing unit includes an imaging-based sensor(s), that can capture a moving image related to blood flow.

In some embodiments, where the sensor(s) is (are) optically based, the light can be transmitted thereto and/or received therefrom via optical fibers.

As the itinerary of the artery may not be readily known without imaging, the one-dimensional sensor array exemplified in FIG. 3 may turn out to be limited in providing the required measured data.

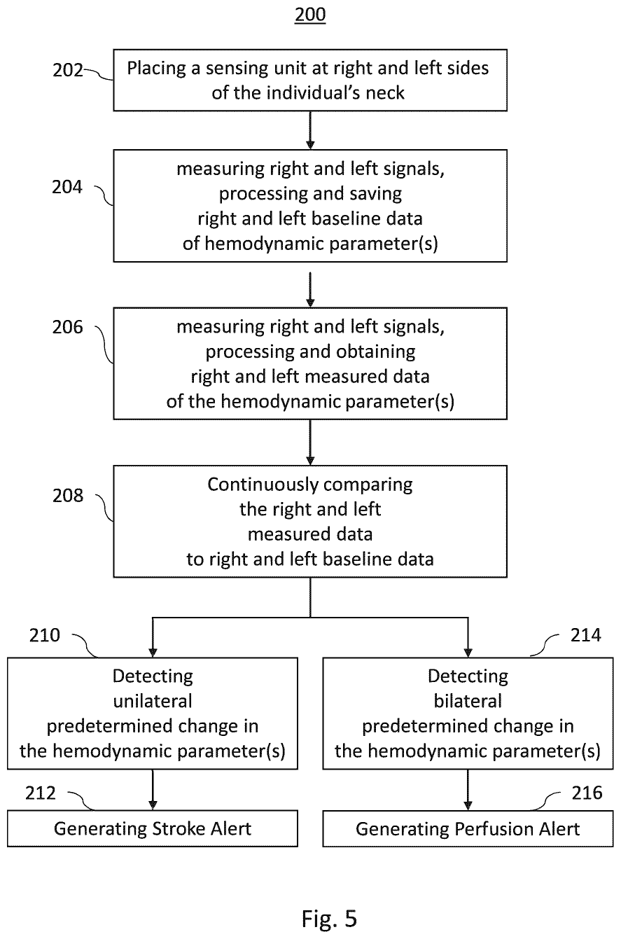

Reference is made FIG. 4 illustrates a non-limiting example of a two-dimensional sensor array 112B, including 6×6=36 sensor elements A1-F6, according to some exemplary embodiments of the invention.

According to some exemplary embodiments, this matrix configuration allows easy placement of sensor 112B on the body (neck) without knowing the exact artery itinerary to place the sensor accurately above the artery. Optionally, at the beginning of monitoring, system 100 optionally collects signals from all the sensors in the matrix, analyzes them, and determines the sensors lying along the path of the monitored artery. In the shown example, signals (e.g., Artery capacitance pulses) can be detected by sensors B5-B6, C1-C6, and D1-D2.

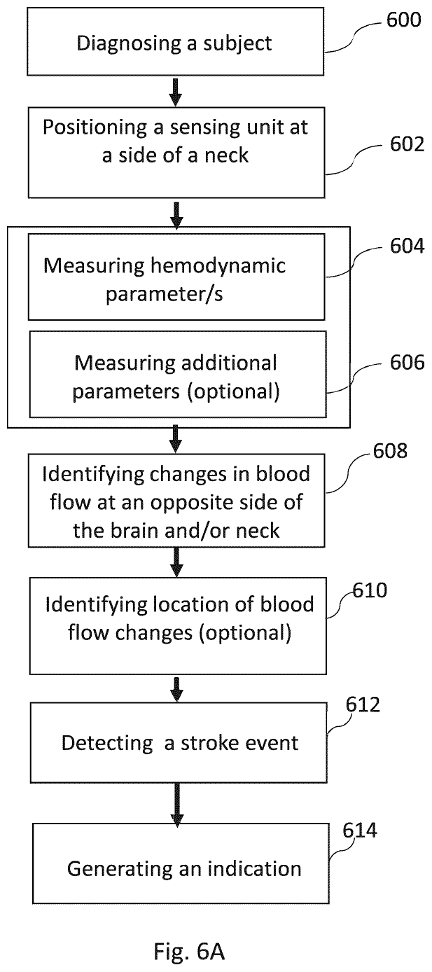

In some embodiments, a non-limiting example of process 200 utilized by system 100 may include the following steps, as illustrated in the flow diagram of FIG. 5, according to some exemplary embodiments of the invention.

In the figure, two scenarios are described, a first scenario is when a change in the hemodynamic parameter(s) occurs on one side of the neck, and the second scenario is when a change in the hemodynamic parameter(s) occurs on two sides of the neck. It is appreciated that these two scenarios are not co-related.

In some embodiments, in step 202, it was selecting the blood vessels to be monitored (e.g., right and left common carotids) and placing corresponding sensing units (each including one or more sensors) in the vicinity. For example, the sensor array described in FIG. 3 or 4 is used to determine the blood flow/velocity (measuring the pulse wave velocity). Also, in some embodiments, and in the described example, the sensor(s) are placed on both the right and left sides of the individual's neck to collect measurements from the two common carotids at least. It is again noted that in another example (not shown) and in some embodiments, the sensor(s) are placed on one side of the neck and configured to collect measurements from the right or left common carotid alone, or from any two blood vessels among the common, external and internal carotids on the same side of the neck.

In some embodiments, in step 204, right and left signals are measured by the right and left sensing units, processing the signals and saving right and left baseline data (personal baseline) of one or more hemodynamic parameters of the common carotids on both sides (left and right) of the neck. In some embodiments, it is assumed that the baseline data refers to the time of a healthy condition of the individual. Optionally, this step is performed once at the start of the monitoring process. Optionally, the baseline data is continuously updated by more measurements. Optionally, the baseline data is obtained from measurements obtained on a plurality of individuals, e.g., based on research and depending on the monitored hemodynamic parameter(s).

In some embodiments, in step 206, continue collecting continuous measurements indicative of one or more hemodynamic parameters from both right and left common carotids and processing the signals to obtain right and left measured data of the hemodynamic parameter(s).

In some embodiments, in step 208, continuously analyzing the measurements by comparing the right and left measured data each to its corresponding baseline data to detect changes from the corresponding baseline data of the individual. Possibly, comparing the continuously received measured data to historical data, including parameters other than the specific hemodynamic parameter(s) being measured. Possibly, the detected changes are above a predetermined threshold.

In some embodiments, Analyzing the differences and comparing is performed, using an algorithm based on historical data and baseline data stored, for example, to find a connection/correlation between these types of differences and the blood supply condition.

In some embodiments, in step 210, if the detected changes are unilateral (on one side of the neck), generating, at step 212, an alert of the possibility of a stroke at the ipsilateral side of the body.

In some embodiments, in step 214, if the detected changes are bilateral happening on both sides, generating, at step 216, an alert of a perfusion problem.

In some embodiments, the analysis of the differences may include a comparison to the baseline data (both personal and collective found in a database).

In some embodiments, the system 100 may be configured to detect continuous and/or discrete changes from the baseline.

In some embodiments, as mentioned above, the invention may utilize other inputs of parameters to generate the output data indicative of the blood supply condition to the brain, for example, one or more of:

### Heart Rate Variability;

Emboli detection—Detection of increased particle flow relative to a baseline in terms of (1) size, (2) volume, and (3) frequency;
Venous drainage—As the stroke evolves, there will be a hemodynamic change in the ipsilateral internal jugular vein compared to (1) baseline, (2) contralateral jugular, and (3) ipsilateral external jugular vein. Therefore, measurement of vein hemodynamics may also be included for corroborating stroke detection;
EEG—As one hemisphere suffers ischemia relative to the contralateral hemisphere, changes in physiological parameters, such as EEG, can be detected compared to a predetermined baseline, such as changes in (1) synchrony and (2) symmetry, both in frequency and amplitude. Therefore, the measurement of EEG may also be included for corroborating stroke detection.

Accordingly, secondary measurements corroborating detection can be used together with the hemodynamic measurement. The secondary measurements can be provided by sensors of the system of the invention or by external sensors communicating with the system.

### Exemplary Process for Detecting Changes in Blood Flow to the Brain

According to some exemplary embodiments, a device positioned on a neck of a subject is configured to measure signals that can be used for detecting a change in blood flow circulation between the body and the brain. In some embodiments, the device, for example, a sensing unit that comprises at least one detector, measures signals that can be used for hemodynamic change in one or more blood vessels between the brain and the brain and the subject body located at a side of the neck that is opposite to the position of the device. In some embodiments, identifying a hemodynamic change in blood vessels at any side of the neck from a single location on the neck allows having a system for detecting a stroke event that is minimized, optionally at least partly implanted, with less interference and complications relative to a system that need to measure signals from both sides of the neck. Additionally or alternatively, the system and/or device described here allows to identifying small and/or frequent hemodynamic changes that may indicate a pending stroke event.

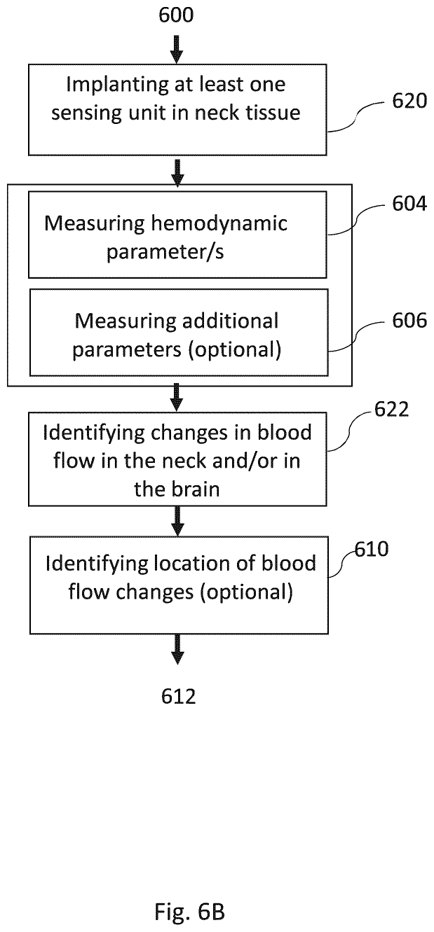

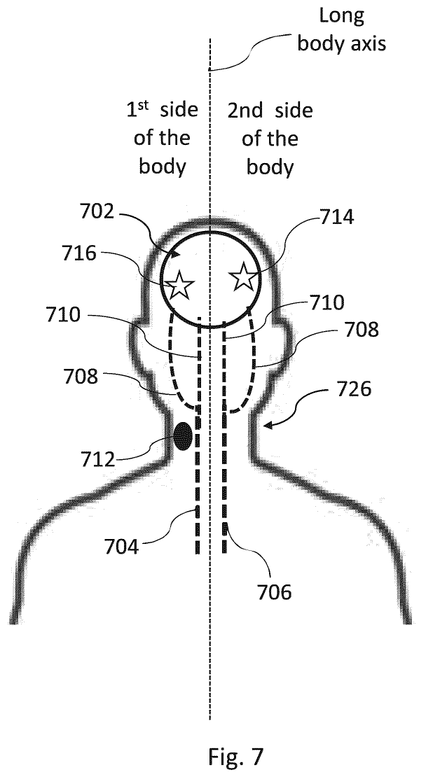

Reference is now made to FIGS. 6A-B, depicting a process for detecting changes in blood circulation in and/or towards a subject brain, according to some exemplary embodiments of the invention.

According to some exemplary embodiments, a subject is diagnosed at block 600. In some embodiments, the subject is diagnosed with a high risk for having an ischemic event, for example, a stroke event. In some embodiments, the diagnosed subject already suffered from at least one stroke event. In some embodiments, the subject is diagnosed with a high risk for recurrent stroke, for example, recurrent ischemic stroke. Alternatively or additionally, the subject is diagnosed with at least one of, high risk for developing stroke, atrial fibrillation, aneurysms, heart diseases, blood vessels diseases, conditions that can cause blood clots, coronary heart disease, heart valve disease, and/or carotid artery disease.

According to some exemplary embodiments, the subject is diagnosed by a health care provider (HCP), for example, a physician, an expert, or a neurologist. In some embodiments, the HCP prescribes or suggests to use of a system for detecting a stroke event, based on the subject diagnosis.

According to some exemplary embodiments, a sensing unit is positioned at a single side of the neck of a subject, at block 602. In some embodiments, the subject is the subject diagnosed at block 600. In some embodiments, the sensing unit is positioned at the left side of the neck, optionally closer to a left carotid artery and the left carotid bifurcation than to the right carotid artery and the right carotid bifurcation. Alternatively, the sensing unit is positioned at the right side of the neck, optionally closer to the right carotid artery and the right carotid bifurcation than to the left carotid artery and the left carotid bifurcation. In some embodiments, the position of the sensing unit is determined based on the subject diagnosis. In some embodiments, a location for placing the sensing unit is selected using ultrasound, for example Doppler ultrasound.

According to some exemplary embodiments, positioning of a sensing unit comprises attaching the sensing unit to a skin surface of the neck, optionally at the left or right side of the neck. Alternatively, positioning of the sensing unit comprises implanting the sensing unit, at least partly into the neck tissue, for example subcutaneously implanting the sensing unit, optionally at the left or right side of the neck. In some embodiments, the positioning of a sensing unit comprises the positioning of a housing that comprises at least one detector, for example, at least one sensor, at least two detectors, at least 3 detectors, or a housing with any larger number of detectors.

According to some exemplary embodiments, at least one hemodynamic parameter is measured at block 604. In some embodiments, the at least one hemodynamic parameter comprises heart rate, heart rate variability, heart contractility, and heart contractility pattern for example using electrocardiogram measurements, arterial pressure, or changes thereof for example using pulse wave measurements.

According to some exemplary embodiments, additional parameters are optionally measured at block 606. In some embodiments, the additional parameters comprise sound. Alternatively or additionally, the additional parameters comprise temperature, for example, the temperature of a region of the subject body, or changes thereof. In some embodiments, some parameters are measured in order to confirm contact with the skin, for example temperature and/or blood flow using a detector, for example a photoplethysmogram (PPG) sensor. Alternatively, contact is determined based on electrical measurements, for example impedance measurements.

According to some exemplary embodiments, the measurements performed at blocks 604 and 606 are performed based on signals received from the sensing unit or at least one detector, optionally positioned at a side of the neck at block 602. Alternatively, the measurements performed at blocks 604 and 606 are performed from both sides of a neck, for example by at least two sensing units.

According to some exemplary embodiments, a change in blood flow, for example, a hemodynamic change, at a contralateral part of the body towards and/or in the brain, is identified at block 608. In some embodiments, the change in blood flow is detected based on the hemodynamic parameters measured at block 604. Optionally and additionally, the change in blood flow is detected based on the measurement of the additional parameters, at block 606.

According to some exemplary embodiments, the change in blood flow is determined based on a determined relation between values of the hemodynamic parameters measured at block 604 and at least one reference value. Optionally and additionally, the change in blood flow is determined based on a relation determined between values of the one or more additional parameters and at least one reference value.

According to some exemplary embodiments, a location of the blood flow changes in the brain and/or in the neck is optionally identified at block 610. In some embodiments, the location of the blood flow changes is optionally identified based on the hemodynamic parameters measured at block 604 and/or based on the additional parameters measured at block 606.

According to some exemplary embodiments, an ischemic event, for example, a stroke event, is detected at block 612. In some embodiments, the stroke event is detected based on the changes in blood flow identified at block 608. Additionally, the stroke event is detected based on the location of the blood flow changes optionally identified at block 610. In some embodiments, the stroke event is determined when the changes in blood flow identified in the brain are not within a range of reference values.

According to some exemplary embodiments, an indication, for example, an alert signal, is generated at block 614. In some embodiments, the indication is a human-detectable indication, for example, an audio and/or a visual indication. In some embodiments, the indication is generated when a change in blood flow in the brain is identified at block 608 and/or when a stroke event is detected at block 614. In some embodiments, the indication comprises information on the hemodynamic change, for example, information on the level of blood flow reduction and/or information on the level of increase of pressure in the blood vessel. Alternatively or additionally, the indication comprises information on a location in the brain of the identified changes in blood flow, for example, the part or region of the brain where the changes in blood flow are located.

According to some exemplary embodiments, the indication comprises instructions to at least one of, the subjects, a caregiver of the subject, and/or instructions to an expert or an HCP monitoring the subject state. In some embodiments, the instructions comprise instructions to take at least one drug, instructions to change body posture, instructions to perform a medical procedure, instructions to change the behavior of the subject, and/or instructions to call emergency services or call for assistance.

According to some exemplary embodiments, the indication generated at block 614 is transmitted to a remote device, for example, a remote computer or a remote server, optionally as part of a telemedicine service for monitoring the state of the subject.

According to some exemplary embodiments, once the controller sets an alert, the patient asked about their condition. In some embodiments, if the patient does not feel well or does not respond, the remote computer/cellphone will alert the telemedicine service selected, providing the provider with the patient's details (for example GPS location and/or the carotid pulse). optionally to enable early evacuation and intervention.

According to some exemplary embodiments, for example, as shown in FIG. 6B, measurements are performed by one or more sensing units implanted into the neck, and the changes in blood flow are identified at one or both sides of the neck.

According to some exemplary embodiments, at least one sensing unit is at least partly implanted in neck tissue, at block 620. In some embodiments, at least one sensing unit is subcutaneously implanted in the neck tissue. Optionally, at least one sensing unit is implanted in the vicinity of a carotid artery in the neck. In some embodiments, two sensing units are at least partly implanted in the neck. Each of the at least two sensing units is implanted at a different side of the neck, optionally near a carotid artery that passes near the implantation site.

According to some exemplary embodiments, the at least one sensing unit is used to measure at least one hemodynamic parameter, for example as described in FIG. 6A.

According to some exemplary embodiments, changes in blood flow in the neck and/or in the brain, are identified at block 622. In some embodiments, the changes are identified in one or both sides of the neck and/or in one or both sides of the brain.

### Exemplary Blood Flow Detection

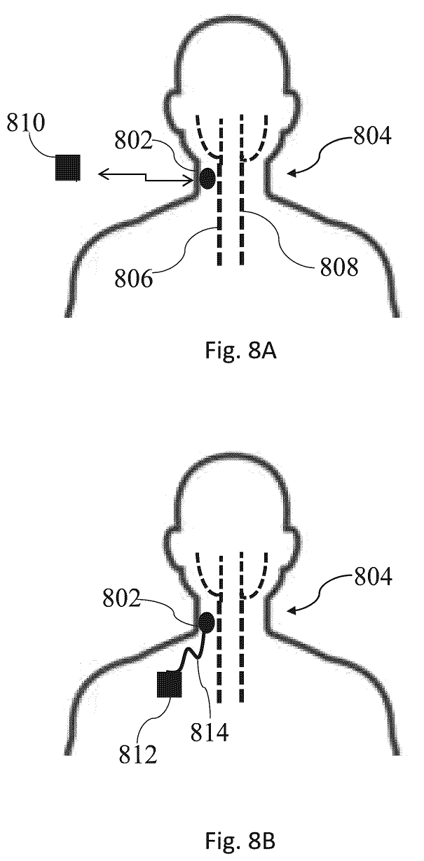

Reference is now made to FIG. 7 depicting the detection of changes in blood circulation, for example, blood flow, in the brain, according to some exemplary embodiments of the invention.

Without being bound by any theory, the brain 702 receives arterial blood supply from a first common carotid artery 704 and a second common carotid artery 706, each is located at a different side of the body. Each of the common arteries is divided at a bifurcation into an external carotid artery 708 and an internal carotid artery 710 extending into the brain.

According to some exemplary embodiments, a sensing unit 712 is positioned on a surface of the neck 726 or is implanted into the neck tissue, at a side of the neck 726, optionally in proximity to one of the common carotid arteries or its extensions. In some embodiments, the sensing unit comprises one or more detectors, for example, a plurality or an array of detectors. In some embodiments, the detectors are configured to measure at least one of, the pulse waves, ECG, sound, heart rate, temperature, body movement, and/or pressure in blood vessels.

According to some exemplary embodiments, the sensing unit 712 is part of a system for identifying changes in blood circulation in the subject brain and is configured to identify changes in blood circulation at a contralateral part of the brain, for example at location 714 which is at an opposite side of the body relative to the location of the sensing unit 712. Additionally, the system is configured to detect changes in blood circulation at an ipsilateral part of the brain, for example at location 716, which is at the same side of the body as the location of the sensing unit 712.

According to some exemplary embodiments, in a case, the identified changes indicate an ischemic event, for example, a transient ischemic event, an acute ischemic event, or a stroke event, the system generates and delivers an indication, for example, an alert signal.

### Exemplary System Positioning

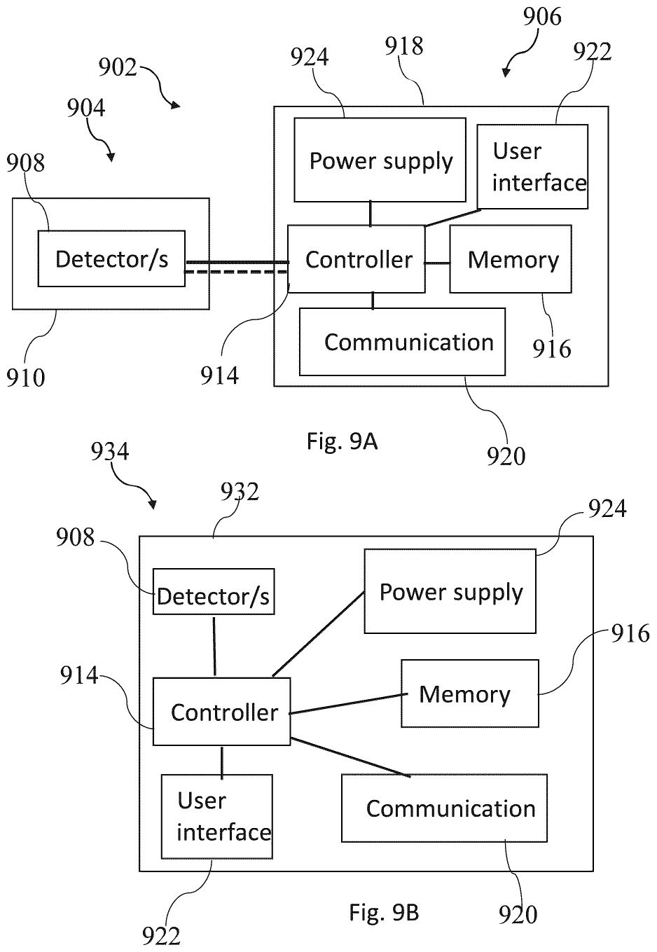

Reference is now made to FIGS. 8A and 8B, depicting the positioning of a device, for example, a sensing unit, at a neck region, according to some exemplary embodiments of the invention.

According to some exemplary embodiments, a sensing unit 802 comprising at least one detector or a plurality of detectors is positioned at a region of a neck 804 in proximity to a first arterial blood vessel 806 delivering blood to the brain of a subject, and at a distance from a second arterial blood vessel 808 delivering blood to the brain located at an opposite side of the neck 804. In some embodiments, for example, as shown in FIG. 8A. the sensing unit 802 is positioned on top of a skin surface of the neck, and is optionally attached to the skin surface via an adhesive and/or a fastener, for example a collar. Alternatively, the sensing unit 802 is implanted, for example subcutaneously implanted at least partially in the neck 804.

According to some exemplary embodiments, for example as shown in FIG. 8A, the sensing unit 802 is in communication with a control unit 810 located outside the subject body. In some embodiments, the control unit is a wearable device fastened to the subject body, for example a hand wrist band or a watch fastened to the subject hand. In some embodiments, the sensing unit 802 is in wireless communication with the control unit, for example communication via Bluetooth, or Wi-Fi, or any other radio waves communication.

According to some exemplary embodiments, for example as shown in FIG. 8B, both the sensing unit 802 and a control unit 812 are implanted, at least partly, in the subject body. In some embodiments, the sensing unit 802 is implanted in the neck 804 of the subject, and the control unit 812 is implanted in the chest of the subject, for example below the collar bone. Alternatively, the control unit 812 is implanted in the back of the subject, in the shoulder of the subject or in the subject neck. In some embodiments, the control unit 812 communicates with the sensing unit 802 via wiring 814 interconnecting the two units. Alternatively, the control unit and the sensing unit are positioned within a single implantable housing, for example a thin, small profile housing configured to be implanted in the neck.

### Exemplary System

According to some exemplary embodiments, a system for monitoring brain blood circulation, for example blood flow in the brain and/or for detecting changes in the blood flow, comprises a sensing unit which includes at least one detector, and a control unit that is in communication with the at least one sensor. In some embodiments, the system is at least partly implantable, for example when the sensing unit is implantable and the control unit can be implantable or positioned outside a subject body. Alternatively, both the sensing unit and the control unit are configured to be positioned outside the subject body.

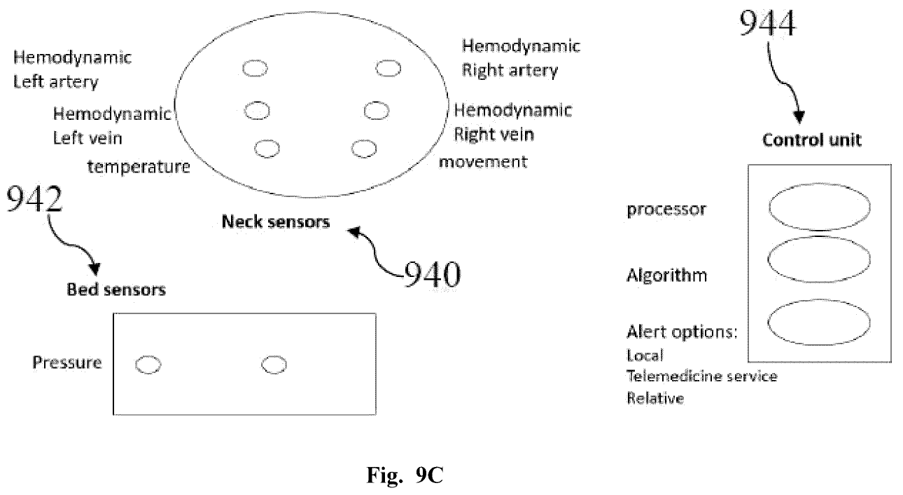

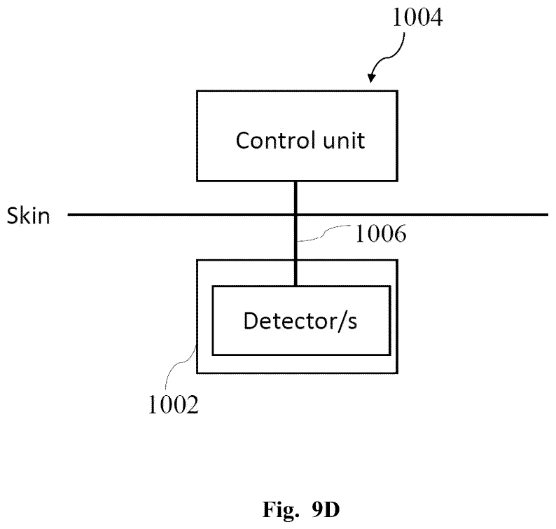

Reference is now made to FIG. 9A, depicting a system for monitoring blood flow in the brain, and/or for detecting changes in the blood flow, according to some exemplary embodiments of the invention.

According to some exemplary embodiments, a system 902 comprises a sensing unit 904 and a control unit 906. Optionally, the system can include two or more sensing units, functionally coupled to a single control unit or to two or more control units. In some embodiments, the sensing unit 904 comprises at least one detector 908, for example a sensor or an electrode. In some embodiments, the at least one detector 908 comprises at least two detectors, at least 3 detectors, at least 4 detectors or any larger number of detectors. In some embodiments, the at least one detector is configured to record electrical signals, for example electrical signals indicating at least one of, heartbeat, movement of blood vessel walls, pulsation of blood vessels, pressure within blood vessels, or changes thereof. Alternatively or additionally, the at least one detector is configured to record sound signals from the tissue. In some embodiments, the at least one detector comprises at least one of, a piezo detector, a pressure detector, a displacement detector, and/or a photoplethysmogram (PPG) detector.

According to some exemplary embodiments, the sensing unit 904 comprises a housing 910. In some embodiments, the housing 910 is configured to seal the at least one detector from body fluids and/or tissue. In some embodiments, the housing 910 is shaped and sized to be implanted in a body tissue, for example in the neck of the subject. In some embodiments, the housing has a thin, low profile casing suitable for implantation, for example subcutaneous implantation. In some embodiments, an outer surface of the housing 910 is flat and smooth, for example to prevent damage to tissue of the body. Optionally, the housing 910 is flexible, for example to conform to anatomy of the body and/or to flex when the neck moves.

According to some exemplary embodiments, in case the sensing unit 904 is configured to be positioned on an outer skin surface of the neck, the sensing unit 904 is shaped as a skin patch, optionally having at least adhesive layer for firmly attaching the sensing unit 904 to the skin surface. In some embodiments, the sensing unit 904 shaped as a skin patch is thin and is optionally flexible, for example to conform to the skin surface of the neck. In some embodiments, the sensing unit 904 shaped as a skin patch has a color which is similar to a color of the skin surface of the subject, for example to reduce visibility of the skin patch when it is attached to the skin surface. In some embodiments, the non-implantable sensing unit 904, optionally shaped as a skin patch is disposable. Optionally, the non-implantable sensing unit 904 is water resistant.

According to some exemplary embodiments, the control unit 906 comprises a controller 914 and a memory 916, positioned inside a housing 918. In some embodiments, the controller 914 receive signals recorded by the at least one detector 908. In some embodiments, the controller 914 processes and/or analyzes the received signals using at least one algorithm, software, formula and/or lookup table, stored in the memory 916. In some embodiments, the control unit 906 comprises a communication circuitry 920, configured to communicate with a remote device, for example a remote computer, a cellular phone, a mobile device, and/or a wearable device. In some embodiments, the communication circuitry communicates with the remote device using wireless signals.

According to some exemplary embodiments, the control unit 906 optionally comprises a user interface 922, configured to generate and deliver human detectable indications, for example an audio and/or a visual indication.

According to some exemplary embodiments, the controller 914 is configured to receive signals from the at least one detector 908, and to process and analyze the signals, for example in order to identify changes in blood flow in the brain, for example in an ipsilateral of the brain or in a contralateral of the brain, for example as shown in FIG. 7. In some embodiments, the controller 914 is configured to identify changes in blood circulation in the brain based on the signals measured by the sensing unit 910 and optionally based on additional measurements.

According to some exemplary embodiments, the controller 914 is configured to determine a stroke event in the subject, based on the identified changes in blood flow or based on a different analysis of the signals received from the at least one detector 908.

According to some exemplary embodiments, in case the control unit 906 is located outside the subject body, the controller 914 signals the user interface 922 to generate a human detectable indication if the identified changes in blood flow are higher or lower relative to a reference, optionally predetermined vale. Additionally, the controller 914 signals the user interface to generate the human detectable indication if a stroke event is detected.

According to some exemplary embodiments, in case the control unit 906 is implanted in the subject body, for example as shown in FIG. 8B, the control unit 906 is configured to signal the communication circuitry to transmit signals to a remote device, according to the identified changes in blood flow and/or when a stroke event is detected. Optionally, the remote device generates a human detectable indication in response to the signals received form the communication circuitry 920.

According to some exemplary embodiments, the control unit 906 comprises a power supply 924, for example at least one battery. In some embodiments, the battery is a replaceable battery and/or a rechargeable battery. In some embodiments, in case the control unit 906 is an implantable unit, the battery is configured to be recharged wirelessly, for example by induction charging.

According to some exemplary embodiments, the control unit 906 is functionally coupled to at least one additional sensing unit, for example to at least one additional sensor. In some embodiments, the controller 914 identifies changes in blood flow and/or determines a stroke event base don the signals received from the sensing unit 904 and from at least one additional sensor or at least one additional sensing unit.

According to some exemplary embodiments, the control unit 906 is configured to be positioned at an operation room, for example to allow monitoring of a subject state during surgery. In some embodiments, the control unit 906 is configured to be functionally coupled to one or more detectors used during surgery, and to provide an alert indication regarding at least one of, hemodynamic changes, a stroke event and/or a pending event in a subject that undergoes a surgical procedure.

According to some exemplary embodiments, in case the control unit 906 is an implantable control unit, a housing 918 of the control unit 906 has an outer surface that is flat and smooth. In some embodiments, the housing 918 is thin, and optionally has a low-profile. Optionally, the housing 918 is flexible. In some embodiments, in case the control unit is configured to be attached to the skin surface, the housing 918 comprises an adhesive for closely attaching the control unit 906 to the skin surface.

According to some exemplary embodiments, for example as shown in FIG. 9B, a device 934 has a housing comprising both the at least one detector 908 and the components of the control unit 906 shown in FIG. 9B. In some embodiments, the device 934 is configured to be implanted at least partly inside the body or to be attached to the skin surface.

According to some exemplary embodiments, for example as shown in FIG. 9C, a system for monitoring blood circulation in the brain and changes thereof, comprises a plurality of sensors located at the neck, for example neck sensors 940. In some embodiments, the neck sensors are positioned on both side of the neck. In some embodiments, sensors positioned at a left side of the neck are configured to measure at least one of, hemodynamics of the left artery, hemodynamics of the left vein, temperature, or changes thereof. In some embodiments, sensors positioned at a right side of the neck are configured to measure at least one of, hemodynamics of a right artery, hemodynamics of a right vein, temperature, or changes thereof.

According to some exemplary embodiments, the system optionally uses information measured by one or more bed sensors 942. In some embodiments, the one or more bed sensors are configured to measure pressure by the subject body, movement and/or temperature.

According to some exemplary embodiments, the system further comprises a control unit 944, functionally coupled to one or more of the neck sensors 940 and/or to one or more of the bed sensors 942. In some embodiments, the control unit 944, is configured to receives signals from the sensors, process the received signals, and analyze the processing results in order to identify changes in blood circulation in the brain, for example changes in blood circulation in a right side and/or in a left side of the brain. In some embodiments, the analysis of the processed signals comprises an analysis for determining if the identified changes indicate an ischemic event. In some embodiments, the processor, for example a controller of the control unit processes and analyzes the signals using at least one of, an algorithm, a model, a formula, a classifier, a neural network and/or a lookup table, stored in a memory of the control unit 944.

According to some exemplary embodiments, the control unit 944 is configured to generate and optionally to deliver an alert signal in case changes in blood circulation are identified and/or when an ischemic event is detected. In some embodiments, the control unit 944 generate and delivers the alert using a user interface, for example a display and/or a speaker of the control unit. Alternatively, the control unit 944 generates and delivers the indication to a remote device, for example a remote device of a telemedicine service. In some embodiments, the remote device generates the alert signal in response to the signals received from the control unit.

According to some exemplary embodiments, the control unit, for example control unit 906, is rechargeable, for example to recharge the power supply 924. In some embodiments, during charging, the control unit 906 continues to receive signals form the sensing unit, and is configured to process, analyze and/or deliver an alert during the charging process.

### Exemplary Sensing Unit(s) Positioning

According to some exemplary embodiments, at least one sensing unit of the system is configured to implanted, for example subcutaneously, into a tissue, for example neck tissue. A potential advantage of an implanted sensing unit may be reduction of noise signals and a more accurate signal detection due to a close proximity of the sensing unit detectors to blood vessels of the neck.

According to some exemplary embodiments, for example as shown in FIG. 9D, a sensing unit, for example sensing unit 1002 is configured to be implanted under the skin, for example subcutaneously implanted. In some embodiments, the control unit, for example control unit 1004 is positioned outside the subject body, and optionally attached to the outer surface of the skin. In some embodiments, the control unit 1004 is coupled to the sensing unit 1002 by wiring, for example wire 1006.

According to some exemplary embodiments, the sensing unit or at least one detector is printed on the skin or under the skin surface, for example as a tattoo.

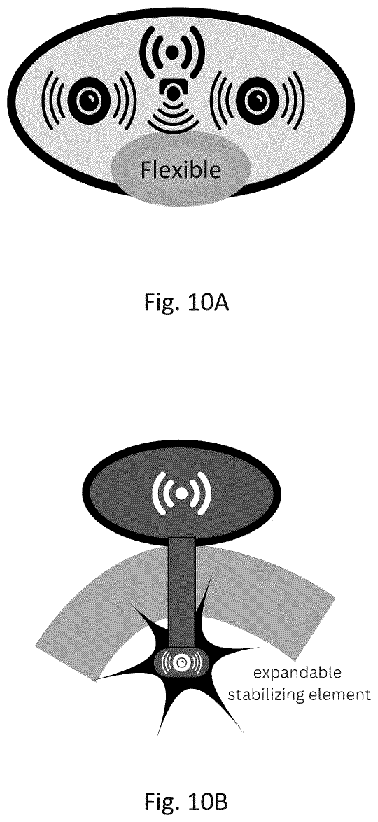

According to some exemplary embodiments, for example as shown in FIG. 10A a device or a sensing unit, is configured to be attached to the skin near one or more carotid arteries. Measurements from a location close to one or more carotid arteries may allow to acquire data about hemodynamic parameters values or hemodynamic changes, that is relevant to the blood supply to the brain. In some embodiments, the device or sensing unit attached to the skin surface is a skin patch that is flexible, for example to conform to the anatomy and curvature of the skin surface. In some embodiments, having at least part of the device or sensing unit flexible, may allow pressure sensing.

According to some exemplary embodiments, for example as shown in FIG. 10B, the device or sensing unit contain an array of sensors with wireless transmission, for example Bluetooth transmission in a single device that can be placed over or under the skin. In some embodiments, the device may have two elements one over the skin and one under the skin with wireless communication in the external portion and the sensors in the internal implanted portion.

According to some exemplary embodiments, the sensing unit is attached to the body or is inserted into body tissue, by subcutaneous implantation, tissue piercing, fixation over the skin using at least one fastener, for example a clip, or a suture, or by sensor printing.

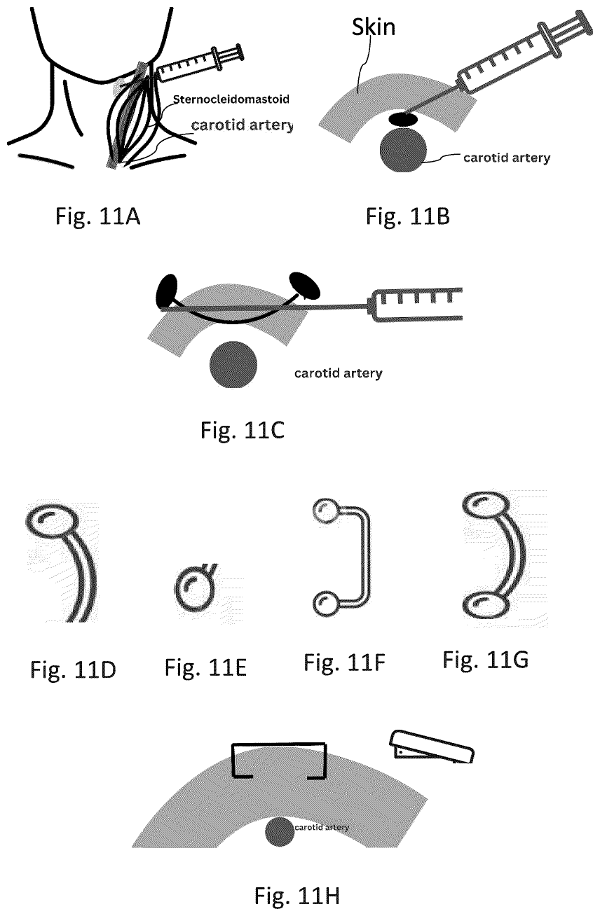

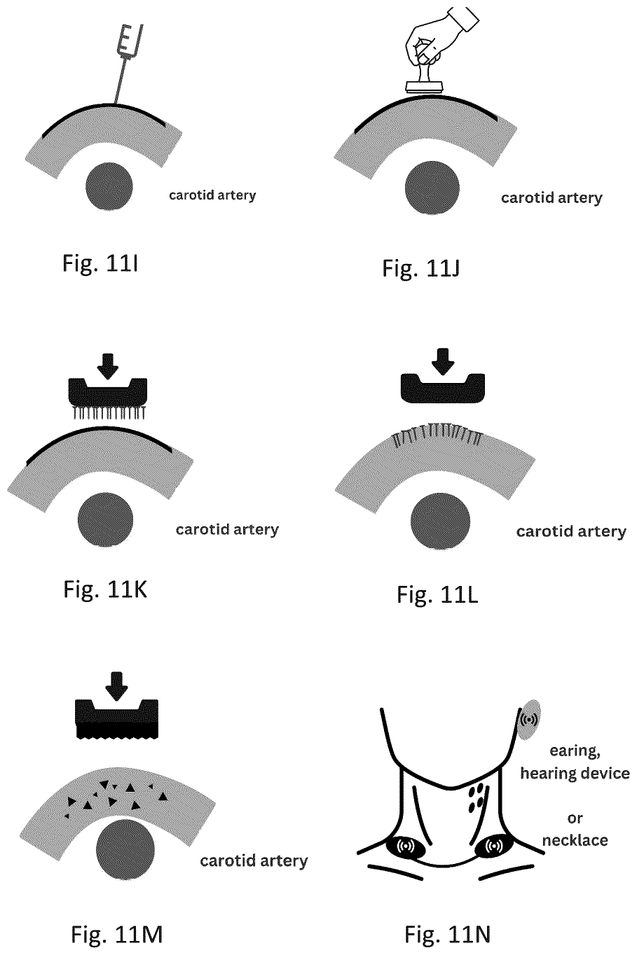

Reference is now made to FIGS. 11A-11N depicting different ways to attach a sensing unit or at least one detector to tissue of the body, according to some exemplary embodiments of the invention.

According to some exemplary embodiments, for example as shown in FIGS. 11A and 11B, a sensing unit or at least one detector is inserted under the skin, subcutaneously, using a syringe. In some embodiments, the sensing unit or the detector is injected under the skin near a Sternocleidomastoideole and in proximity to a carotid artery. In some embodiments, a syringe-like insertion device will be used to insert the device, for example a sensing unit, a control unit, a device which includes both a sensing unit and a control unit, under the skin via a hollow syringe. In some embodiments, the device comprises an expandable element, for example to better adhere the sensing unit to the skin surface and/or to increase a contact area between the sensing unit and the skin to optionally increase sensitivity and/or accuracy of the measurements.

According to some exemplary embodiments, the device, for example the sensing unit, the control unit, both the sensing unit and the control unit and/or at least one sensor, via piercing into the tissue, for example into neck tissue. In some embodiments, for example as shown in FIGS. 11D-11G the device comprises 2 elements, a piercing (FIG. 11D) Piercing (a) and a closure element (FIG. 11E). FIGS. 11F and 11G show different shapes of a piercing element. In some embodiments, the piercing element is inserted through two adjacent skin points, and then is closed by the closing end shown in FIG. 11E, while the device or sensing unit is attached to the skin surface.

According to some exemplary embodiments, for example as shown in FIG. 11H, the device is fastened to the skin using staples, for example in close proximity to a carotid artery.

According to some exemplary embodiments, for example as shown in FIGS. 11I-11M, the device is printed or is at least partly inserted or implanted into the skin.

According to some exemplary embodiments, a skin-interfaced, wearable electronics, bio-integrated sensors with optionally wireless transmission modules are printed on the skin with a designated printing device. In some embodiments, the sensors include printed sensors or any device that detects and monitors hemodynamic flow. In some embodiments, the sensors include direct sensors or sensors that include a tie layer, for example Piezoelectric Poly (vinylidene fluoride) (PVDF-based sensors, strain gauge based sensors, and/or capacitor based sensors. In some embodiments, the sensors include indirect sensors, for example sonar based sensors, photoplethysmogram (PPG) based sensors, infrared based sensors, and/or magnetic sensors.

According to some exemplary embodiments, the device or one or more sensors are tattooed with a needle (FIG. 11I), printed/stamped/applied on the skin surface (FIG. 11J), pierced into the skin (FIGS. 11K and 11L), nano sensors that are reach extra cellular and/or intracellular (FIG. 11M). In some embodiments, for example as shown in FIG. 11N, the sensors in the skin with one or more external device, for example wearable devices such as a necklace, an earring or a hearing device.

According to some exemplary embodiments, integrated systems and/or soft body area sensor networks include on-body sensors for hemodynamic monitoring and flexible printed circuit boards for signal conditioning/readout and wireless transmission. In some embodiments, printed electronic sensors on the human body can be designed as follows: Intercellular sensors (micro and nano levels) assembled or self-assembly conductive coating (for instance: dispersion, emulsion, suspension, or other) that utilize as an ink source to produce the printed circuit, hence sensors. In some embodiments, the chemical coating may contain nano/microparticles utilized as a conductive coating or dispersion material to create a sub or outer sensors patterned layer. Optionally utilizing solvents as carrier phase (conductive particles or micro/nano sensors) to etch a stratum corneum layer of the skin or any outer layer of the skin, allow for example, controlled and/or gradual penetration into the skin.

### Exemplary Monitoring Method Using an Exemplary Model

According to some exemplary embodiments, a method for monitoring brain blood flow or changes thereof, is used for detecting a stroke event.

According to some exemplary embodiments, the method uses at least one of, pulse wave measurements, ECG measurements and measurements of acoustic signals, for example by one or more sensors of a sensing unit, as previously described. In some embodiments, the system comprises different types of sensors, for example at least one acceleration/pressure sensor, at least one flow-indicating sensor (PPG), at least one sensor for measuring an Electrocardiogram (ECG) signal, and/or a microphone. In some embodiments, acoustic (microphone) and blood flow (pulse wave) sensors are located on the left or right side of the neck. Alternatively, the sensors are located at both sides of the neck. In some embodiments, pulse waves are measured using at least one of, at least one piezo sensor, at least one pressure sensor, at least one accelerometer, at least one photoplethysmogram (PPG) sensor and/or at least one acoustic sensor, for example a microphone.

According to some exemplary embodiments, the pulse wave configuration, together with an ECG sensor, measures the pulse wave propagation time between the heart and carotid, which is an indicator of cerebral blood flow. In some embodiments, the acoustic sensor detects subtle sound changes in the carotid artery caused by the turbulent blood flow associated with stenosis or occlusion. In some embodiments, the processor, for example a controller, processes the data from the sensors and compares it with a reference database to detect abnormalities indicative of a stroke.

According to some exemplary embodiments, the system and method first capture the pulse wave signals from the sensors and preprocess them to remove noise and artifacts. In some embodiments, the raw signals are then passed through a signal processing feature extraction which extracts morphological and time-based features from the pulse wave signals. In some embodiments, the morphological features include at least one of, waveform amplitude, frequency, and/or shape, while the time-based features include time intervals between specific points in the waveform. In some embodiments, the raw signal itself, together with the features, are introduced into a deep learning architecture to detect potential disruptions or changes in blood flow, indicating the presence of a blockage or obstruction in the carotid artery. Alternatively or additionally, the “deep learning architecture” detects changes in blood circulation in the brain. In some embodiments the measurements of the signals, the processing and the analysis are performed continuously. In some embodiments, the output of the learning architecture/model is stored, for further analysis.

According to some exemplary embodiments, the system and method can adapt to different patterns in pulse wave signals and improve detection accuracy. In some embodiments, the system and method include LSTM layers and a self-attention mechanism, for example to allow the system to work with raw pulse signals, to optionally increase the sensitivity and specificity of the detection. In some embodiments, an array of 2, 3, 4, or any larger number of pulse wave sensors, optionally aligned along the carotid artery of the neck, is used, for example to increase a reliability of the system and method.

The optionally non-invasive, and continuous carotid monitoring system and method provide an early warning to physicians, allowing for timely intervention and prevention of severe health complications. In some embodiments, the system is cost-effective and can be used in various healthcare settings, improving patient outcomes and reducing healthcare costs.

Reference is now made to a general method for detecting of an ischemic event, for example a stroke event, according to some exemplary embodiments of the invention.

According to some exemplary embodiments, at least one ECG signal is optionally measured at block 1202. In some embodiments, the ECG signals is measured by at least one ECG sensor in a sensing unit of a device.

According to some exemplary embodiments, at least one pulse wave is measured at block 1204. In some embodiments, at least 2, for example 3 pulse waves (3 measurement channels), are measured at block 1204. In some embodiments, the pulse waves are measured by at least one of, at least one piezo sensor, at least one pressure sensor, at least one accelerometer, at least one photoplethysmogram (PPG) sensor and/or, optical sensor, strain gauge, magnetic sensor, fiber optic, laser, and/or at least one acoustic sensor, for example a microphone.

According to some exemplary embodiments, different features are extracted from the measured ECG and pulse wave signals, at block 1206. In some embodiments, the features comprise morphological features and/or time based features. Alternatively, features are extracted only form the pulse wave signals, for example when ECG is not measured.

According to some exemplary embodiments, acoustic signals are optionally measured at block 1208. In some embodiments, the acoustic signals are measured by at least one microphone in the sensing unit of the device or system. In some embodiments, the acoustic signals are measured during the measurements performed at blocks 1202 and/or 1204

According to some exemplary embodiments, at least one feature is optionally extracted from the acoustic signal, at block 1210.

According to some exemplary embodiments, a quality of the measured signals is optionally determined at block 1212.

According to some exemplary embodiments, a model is applied at block 1214. In some embodiments, the model is applied using the extracted features as input data to the model, for identifying changes in brain blood circulation and/or for identifying changes in blood flow to the brain in the carotid arteries. In some embodiments, information on the signal quality optionally determined at block 1212 is used as input data for the model. Alternatively or additionally, measurements of the acoustic signals and/or features extracted from the acoustic signals are used as input for the model. In some embodiments, the model is applied to determine if the identified changes indicate an ischemic event in a contralateral side or in an ipsilateral side of the brain. Optionally, input data inserted into the model is used to train the model, and optionally to generate models that are personalized to a specific subject, a group of subjects, models that are specific for a specific state, for example a clinical state of a subject, age and/or gender of a subject. In some embodiments, the model comprises a learning model, for example a deep learning model.

According to some exemplary embodiments, a score is received from the model, at block 1216. In some embodiments, the score is calculated by the model based on the input data introduced into the model. In some embodiments, the score is a score indicting a probability for having an ischemic event.

According to some exemplary embodiments, an ischemic event is detected or not, at block 1218. In some embodiments, the system determines if an ischemic event is detected or not based on the score calculated and received from the model, at block 1216.

According to some exemplary embodiments, if an ischemic event is detected, an indication, for example an alert signal is generated and delivered, at block 1220. According to some exemplary embodiments, if an ischemic event is not detected, then the process and blocks 1202, 1204, 1206, 1208, 1210, 1212, 1214, 1216 and 1218 are repeated.

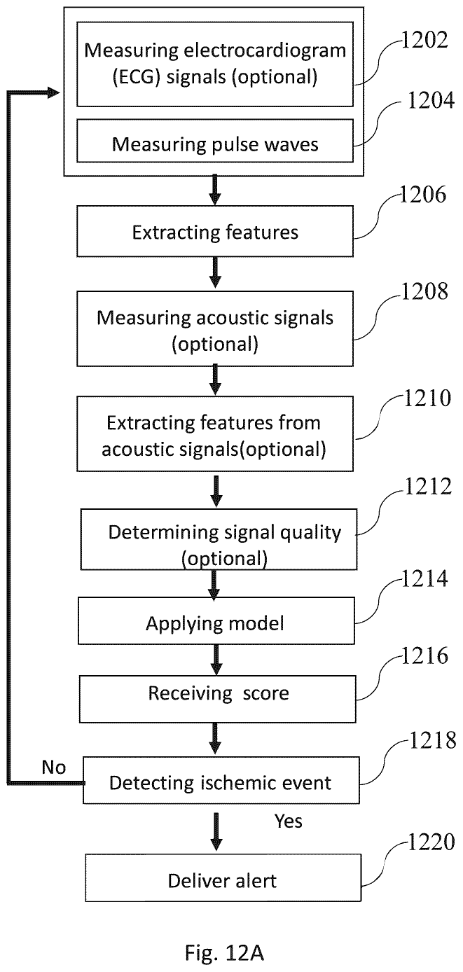

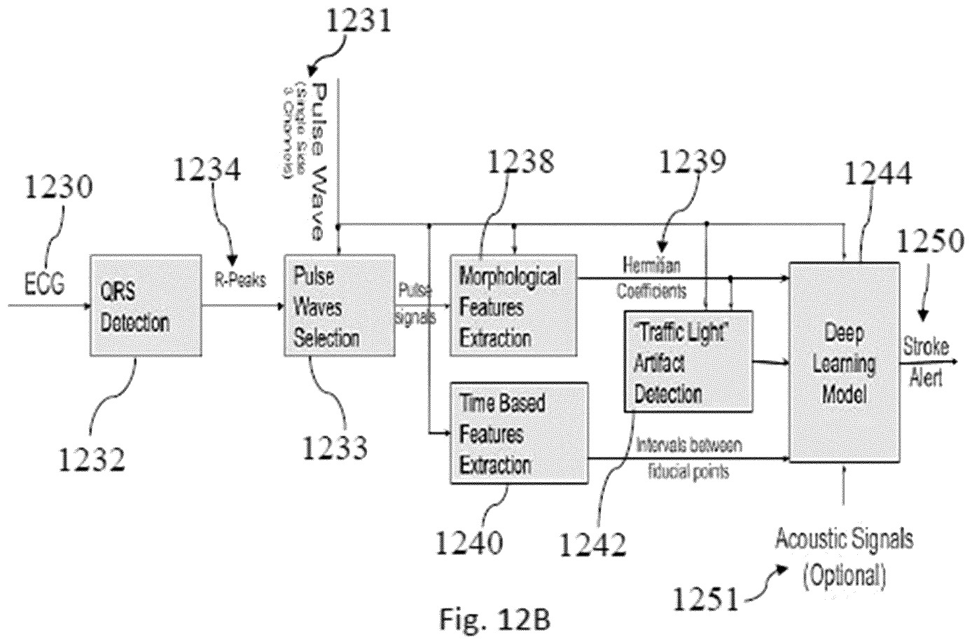

Reference is now made to FIG. 12B, depicting a detailed method for detecting an ischemic event, according to some exemplary embodiments of the invention.

According to some exemplary embodiments, at least one ECG signal 1230 and one or more, for example 2 or 3 pulse waves 1231 are measured. A real-time QRS detection algorithm 1232 (for example Pan & Tompkins, 1985) is applied, for example to detect R wave peak locations 1234. In some embodiments, the R peak locations 1234 are then used to segment the pulse wave signals into individual pulses. Next, pulse signals are processed to extract morphological features 1238 and time-based features 1240. In some embodiments, the morphological features 1238 are processed to detect artifacts using a model, for example a “traffic light” model 1242, and the raw data and raw features are passed through a stroke detecting model, optionally a learning model, for example a deep learning model 1244 to provide stroke alerts.

According to some exemplary embodiments, one or more measured acoustic signals 1251 are used as an input data for the model, for example deep learning model 1244.

According to some exemplary embodiments, the ECG measurements 1230 are used to identify a time window in which measured pulse waves should be further processed, which means that specific pulse waves measurements are selected at block 1233 for further measurements.

### Time-Based Feature Extraction

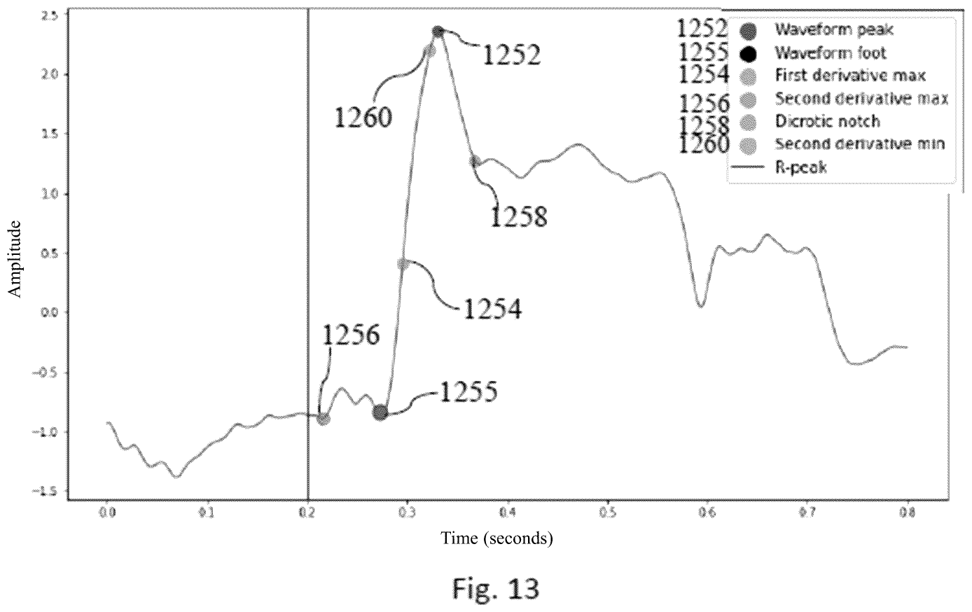

According to some exemplary embodiments, one or more of, putative peaks, shown in FIG. 13—waveform max, waveform foot, first derivative max, second derivative max, and second derivative min peaks—were identified based on Nabeel, Kiran, Joseph, & Abhide, 2020. Optionally or additionally, a dichotic notch peak was identified based on Balmer, Pretty, Amies, Deasaive, & Chase, 2018.

According to some exemplary embodiments, based on putative pulse peak's location, and R wave location, at least one or all of the 20 time interval based features are extracted:
1. Time interval between R wave and Waveform max peak
2. Time interval between R wave and waveform foot,
3. Time interval between R wave and first derivative max,
4. Time interval between R and second derivative max
5. Time interval between R and second derivative min
6. Time interval between R and dichotic notch
7. Time interval between waveform max and waveform foot
8. Time interval between waveform max and first derivative max,
9. Time interval between waveform max and second derivative max
10. Time interval between waveform max and second derivative min
11. Time interval between waveform max and dichotic notch
12. Time interval between waveform foot and first derivative max
13. Time interval between waveform foot and second derivative max
14. Time interval between waveform foot and second derivative min
15. Time interval between waveform foot and dichotic notch
16. Time interval between first derivative max and second derivative max
17. Time interval between first derivative max and second derivative min
18. Time interval between first derivative max and dichotic notch
19. Time interval between second derivative min and second derivative min.
20. Time interval between second derivative min and dichotic notch

In some embodiments, since at least 2 sensors, for example at least 3 sensors are located on one side of the neck, a set of confidence time-based features is calculated using the standard deviation and the mean value of the extracted time interval based features, for example the 20 extracted features. Hence, obtaining 40 features (20 mean time interval features and 20 standard deviation features per pulse wave recording).

In some embodiments, additional features, for example 20 additional features, that are the ratio between the lateral and contralateral mean time interval features, are extracted.

In some embodiments, for example to capture the subtle changes in pulse waves formed due to stroke from a single side array of pulse waves, a set of ratios of the mean and ratio of standard deviation is calculated:
40 ratio features between pulse wave channel 1 and pulse wave channel 2
40 ratio features between pulse wave channel 1 and pulse wave channel 3.
40 ratio features between pulse wave channel 2 and pulse wave channel 3

The overall calculation results with 240 time-based features: 20×3 mean time interval features, 20×3 standard deviation time interval features, and 120×1 ratio of the time intervals. In some embodiments, these features indicate obstruction of blood flow since they quantify the following concepts: Returning Wave, Location of wave change, Timing of wave change, and Pressure vs. flow changes.

### Morphological Feature Extraction

In some embodiments, each pulse velocity wave is decomposed into Hermite basis functions, and the resulting coefficients, for example coefficients 1239 shown in FIG. 12B, and width parameters are used to represent the pulse. In some embodiments, this method is applied for clustering QRS complexes (Lagerholm, Peterson, Braccin, Edenbrandt, & Sörnmo, 2020). In some embodiments, by means of this representation of pulse wave velocity, supervised learning is applied to detect artifacts, and by calculating ratios between lateral and contralateral coefficients, we obtain another set of features indicative of a stroke.

In some embodiments, each pulse wave in a time window between 100 msec and 600 msec, for example in a 400 msec window (configurable) is centered at the location of its max peak, and is modeled as a combination of Hermite basis:

$$( 1 ) x ( t ) = {\sum }_{n = 0}^{N} {c}_{n} ( σ ) \cdot {H}_{n} ( t , σ )$$

$$L ) x ( t ) = {\sum }_{n = 0}^{N} {c}_{n} ( σ ) \cdot {H}_{n} ( t , σ ) + e ( t , σ )$$

H—denote Hermitian polynomials, The first six polynomial basis is depicted in FIG. 3. N—order of Hermite basis it is a configurable number between 6-24, ασ—width of the basis and cₙ(σ)=Σₜx(t)·Hᵤ(t,σ):Σₜx(t)·Hₙ(t,σ): are the coefficients. The parameters cₙ(σ), and σ are selected to minimize the error:

$$( 2 ) {\sum }_{t} {\vert e ( t , σ ) \vert}^{2} = {\sum }_{t} {\vert x ( t ) - {\sum }_{n = 0}^{N} {c}_{n} ( σ ) \cdot {H}_{n} ( t , σ ) \vert}^{2}$$

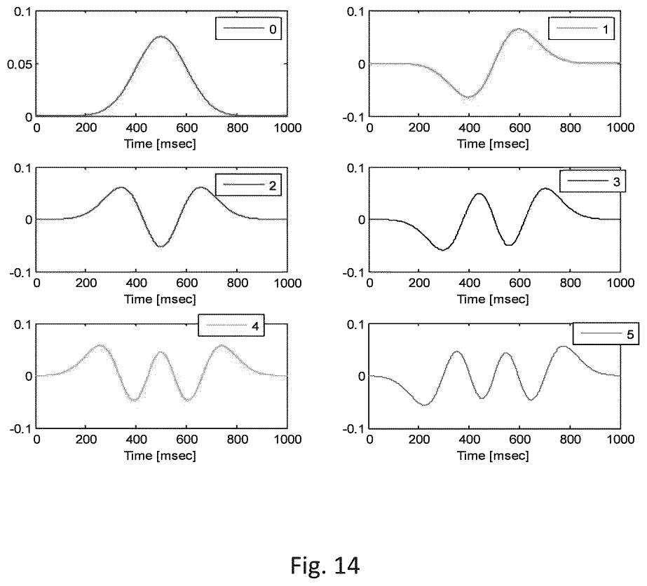

FIG. 14 depicts the first six Hermite basis function.

In some embodiments, each pulse wave is divided or processed by employing a series of coefficients that amplify orthonormal basis functions, for example, Hermite. The initial six Hermite basis functions are illustrated in FIG. 14.

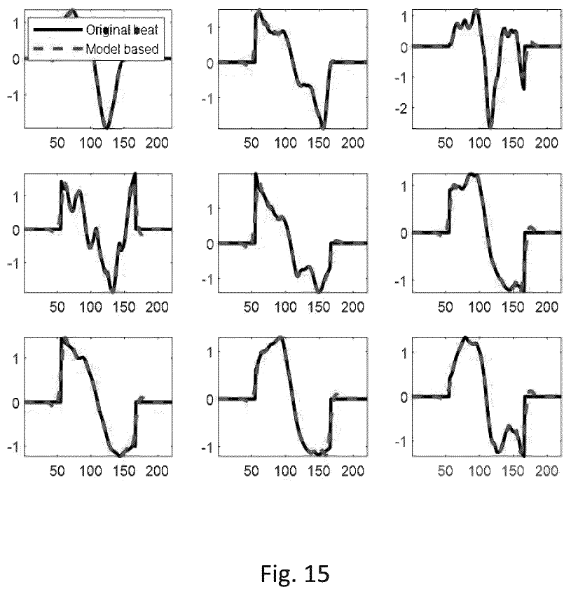

An example of modeling of different waves with Hermite basis function (N=8) for reconstruction of different waves with a set of coefficients is depicted in FIG. 15.

In our exemplary model, and in some embodiments of the invention, N=8N=8 hence, 10 features per pulse wave per channel are obtained. (8 coefficients, σσ and e—error of estimation that is highly indicative of noisy pulse.

In some embodiments, mean and standard deviation of those features over a time window in a range between 1 second and 20 seconds, for example a time window of 10 seconds (configurable) is calculated to obtain total of 20 features per channel.

In some embodiments, 20 additional features that describe ratio between channel 1 and channel 2 features, are then extracted. Additional ratio features from channel 1 and channel 3 and from channel 2 and channel 3 are calculated. Overall, 120 morphological features are obtained: 10×3 mean morphological features, 10×3 standard deviation of morphological features and 60×1 ratio of morphological features.

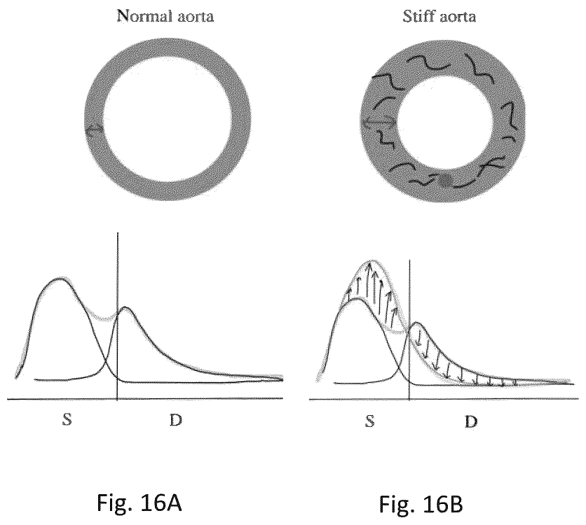

In some embodiments, morphological and time-based features are optionally used for capturing the following clinical insights:
Returning wave: In some embodiments, a pressure wave signal comprises a forward wave and a returning wave. When a blockage or disruption occurs, the returning wave changes its position relative to the measurement before the blockage, according to Laplace's law, and therefore the pressure wave changes its shape. The flow wave also changes in proportion, so the flow speed decreases. Ongoing monitoring of blood flow and pulse wave properties makes it possible to detect changes in the waveform that indicate a blockage or disruption.
Location of wave change: In some embodiments, when a blockage or disruption occurs, a returning wave is generated at the location of the blockage/disruption and progresses backward toward the carotid artery. Optionally, the closer the blockage/disruption is to the carotid artery, the faster the returning wave reaches it. Therefore, the location of the waveform change within the recorded pulse wave cycle may indicate the distance between the obstruction site and the sensors. Additionally, a location of the a change in the cycle in each sensor is correlated with a distance information. Meaning, the Change appears earlier in the sensor cycle closer to the brain and later in the sensor closer to the heart.
Timing of wave change: In some embodiments, a change will begin and become more prominent on the ipsilateral side. Changes will occur later and to a lesser extent on the contralateral side. Optionally, the contralateral side may react by increasing stiffness. FIGS. 16A and 16B show timing of wave change: increasing stiffens, for example when an aorta changes from a normal aorta (FIG. 16A) to a stiff aorta (FIG. 16B), changes flow and pressure waves in different ways. Thus, with increased stiffness the gaps between the flow an pressure wave change (increase or decrease). This also changes the time appearance of fiduciary points.
Pressure vs. flow changes: In some embodiments, by comparing the two waveforms from two different types of sensors, for example one representing the flow and the other pressure, the changes may be different. Optionally, in an event of a blockage, a delta between the waveform measured by the pressure sensor and the waveform measured by the current sensor increases relatively significantly (because the flow decreases compared to a typical waveform. And therefore, the waveform that will result from a pressure sensor following the blockage is higher). In some embodiments, by analyzing the extracted features, the system can determine if there is a blockage or other disturbance in the carotid artery.

### Exemplary Artifact Detection

According to some exemplary embodiments, for example as shown in FIG. 12B, an artifact detection model, for example a “traffic light” model.

In some embodiments, the model, for example the traffic light model is based on a Convolutional Neural Network (CNN) that can detect the quality of pulse wave signals. In some embodiments, the CNN is designed to classify the pulse wave signals into three quality categories: low-red light, moderate-yellow light, and high-green light. The architecture of the traffic light model is optionally given by:
Input layer: The input layer takes in the raw pulse wave data and a set of Hermitian coefficients Morphological features of the pulse wave.
Convolutional layers: the raw pulse wave is fed into a series of convolutional layers, which extract features from the input data using filters of different sizes. The output of each convolutional layer is passed through a rectified linear unit (ReLU) activation function to introduce non-linearity.
Pooling layers: After each convolutional layer, a max-pooling layer is added to reduce the spatial dimensions of the output and increase the computational efficiency of the network.
Fully connected layers: The output of the final pooling layer is flattened and fed into a series of fully connected layers, which combine the extracted features from the CNN and 10 Morphological features from each pulse to make a prediction about the quality of the pulse wave signal. The output of the last fully connected layer is passed through a SoftMax activation function to obtain a probability distribution over the three categories.
Output layer: The output layer consists of three neurons, each corresponding to one of the three categories of pulse wave quality. The neuron with the highest activation is taken as the predicted category.
Training: The network was trained using a labeled dataset of 1,000,000 pulse wave signals, that were manually annotated by trained technicians into three quality categories. The training process involved optimizing the weights of the network to minimize categorical cross-entropy loss function.
Evaluation: The performance of the network was evaluated on a holdout dataset of pulse wave signals that were not used in the training process. For example, the evaluation metrics F1 score was set to be above 0.85 for all three categories.

### Deep Learning Architecture

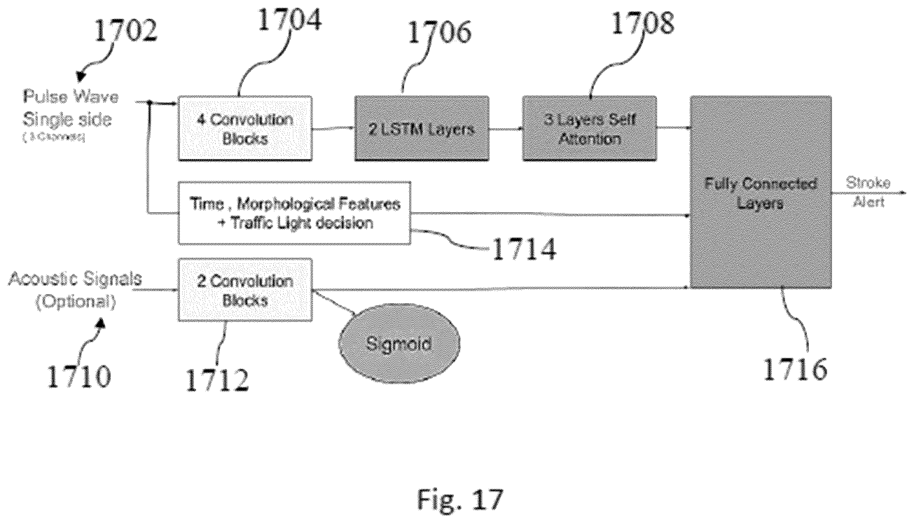

According to some exemplary embodiments, for example as shown in FIG. 12B, a model or a learning model, for example a deep learning model 1244 is used. In some embodiments, the model combines convolutional neural network (CNN), Long Short-Term Memory (LSTM) layers and self-attention mechanism, for example to predict the occurrence of stroke based on input data of 3 pulse velocity signals, set of morphological and time based features and optionally an acoustic signal, for example as shown in FIG. 17.

In some embodiments, the raw pulse waves 1702 are processed through convolution blocks, for example 4 convolution blocks 1704, each containing a convolution layer, batch normalization, Leaky Relu activation function, and max pooling operation, for example to extract high-level features. In some embodiments, an output of the convolutional layer is then processed using Representation Learning Stage that is composed of two LSTM layers 1706 followed by three self-attention blocks 1708, which learn to weight the importance of different parts of the input data based on their relevance to the task, which is detection of an ischemic event in the brain, for example detection of stroke.

In some embodiments, the raw pulse 1702 is also processed, for example as described in FIG. 12B, to obtain morphological features, time-based features and the quality of the pulse waves based on the traffic light model.

In some embodiments, acoustic signal turbulent changes are detected. In some embodiments, microphone-based acoustic signal measurement near the carotid artery are used to measure changes in turbulence, for example due to obstruction in blood flow. This technique is called carotid artery auscultation.

In some embodiments, during this procedure, a microphone is placed on the neck over the carotid artery. In some embodiments, the sound of blood flowing through the artery is amplified by the microphone, and is analyzed to detect changes in turbulence caused by an obstruction in the artery. Optionally, the turbulence caused by the obstruction creates a whooshing or bruit sound that can be detected and analyzed to determine the severity of the stenosis. The CNN-based classifier is trained based on acoustic signals to detect obstruction in blood flow. The acoustic signal 1710 is passed through convolution blocks, for example two convolution blocks 1712, each containing a convolution layer, batch normalization, Leaky Relu activation function, and max pooling layer. The final layer is based on a single sigmoid neuron to predict the obstruction in blood flow. Therefore, an output of the pooling layer will obtain information on turbulence changes.

According to some exemplary embodiments, the output of the representation learning stage (1706 and 1708), morphological, time-based, and quality features (1714), and the output of the pooling layer from the last convolutional layer of the acoustic signal (1712) are passed through fully connected dense layers with at least one, for example 3 sigmoid neurons, to predict an obstruction in blood flow, depth of the obstruction in mm and side of the obstruction.

In general, training of the deep learning model for detecting stroke included the following steps:

### 1. Data Collection and Preprocessing:

a) Collect a large dataset of subjects using our sensor: record pulse velocity wave (PWV), ECG, and audio recordings. The dataset should include both healthy individuals and those who have experienced a stroke.
b) Preprocess the data by performing tasks such as noise reduction, filtering, and normalization to ensure consistency across all three types of data.
c) Extract relevant features from each data type. (We gave a detailed explanation on this section in the patent).

### 2. Model Selection and Development:

a) We gave a general architecture in the patent description, but we will need to fine tune this model by selecting different number and type of layers and number of neurons in each layer.
b) Divide the preprocessed and labeled dataset into training, validation, and testing sets, following an appropriate ratio (e.g., 70% training, 15% validation, and 15% testing).

### 3. Model Training:

a) Train the model using the training dataset by feeding the PWV, ECG, and microphone features as inputs, and the binary labels as outputs.
b) Monitor the training process and fine-tune hyperparameters that minimize validation loss. The following parameters will be tuned: type of loss function, type of optimization, level of regularization, learning rate, batch size, and number of epochs in the training process.

### 4. Model Evaluation:

Evaluate the trained model using the testing dataset by calculating F1-score, sensitivity, specificity, negative predictive value and positive predictive values
Below is a specific process used for development and training of the stroke detecting model.
Training: The network was trained using 5 subjects with labeled segments of pulse wave signals and acoustic signals. There are 4 types of labels:
   Is there an obstruction?
   Depth of the obstruction in mm
   Side of the obstruction
   The depth of the obstruction as was measured neurophysiologist.
The database contained 1581 segments of 1 second of documented obstruction, side, and depth of the obstruction and 1581 segments of 1 seconds without an obstruction. The labeled data used to optimize the weights of the network and to minimize cross-entropy loss function.
Evaluation: The performance of the network was evaluated on a holdout dataset of pulse wave signals, and acoustic signal that were not used in the training process. The evaluation metrics F1 score above 0.9 was observed.

### Evidence of Unilateral Detection

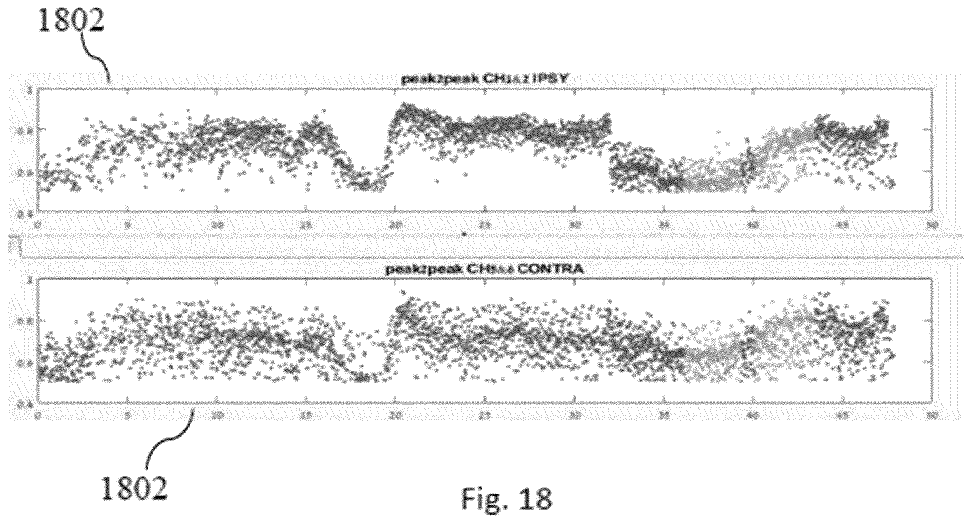

Signal analysis: during this procedure there was block created by the catheter (not balloon blocking the MCA ~17 minutes. As shown for example in FIG. 18, the analysis revealed a drop in peak-to-peak time, (corresponding to a rise in PWV), in both the ipsilateral side 1802 and the contralateral side 1804 (less robust).

Machine learning: the Algorithm was applied on data from one side only. Once with data from the ipsilateral side and once with data from the contralateral side. Both yielded identification of the obstruction however the contralateral side was less robust. (0.684 vs 0.808; 2 tailed t-test=0.19), for example as shown in Table A below:

| | Metric | | |
|---|---|---|---|
| | Precision | Recall | |
| Test Participant | Block \| No Block | Block \| No Block | Accuracy |

Classification report - Ipsi

| | | | |
|---|---|---|---|
| BE001 | 0.98 \| 1.00 | 1.00 \| 0.98 | 0.99 |
| BE002 | 0.75 \| 0.71 | 0.77 \| 0.68 | 0.73 |
| BE003 | 1.00 \| 0.61 | 0.35 \| 1.00 | 0.67 |
| BE005 | 1.00 \| 0.90 | 0.89 \|1.00 | 0.94 |
| GMC009 | 0.74 \| 0.69 | 0.78 \| 0.65 | 0.71 |

Classification report - Contra

| | | | |
|---|---|---|---|
| BE001 | 0.65 \| 0.65 | 0.66 \| 0.64 | 0.65 |
| BE002 | 0.00 \| 0.48 | 0.00 \| 0.91 | 0.45 |
| BE003 | 0.70 \| 0.58 | 0.42 \| 0.82 | 0.62 |
| BE005 | 1.00 \| 0.90 | 0.89 \|1.00 | 0.94 |
| GMC009 | 0.84 \| 0.71 | 0.65 \| 0.87 | 0.76 |

### Exemplary Subject/Patient Management

According to some exemplary embodiments, the system described in this application are used in order to provide continuous monitoring of brain blood circulation. In some embodiments, the system monitors the brain blood circulation by monitoring hemodynamic changes. In some embodiments, the system monitors the brain blood circulation of the subject, when the subject is at home and/or when the subject is engaged in everyday activities. In some embodiments, the system monitors the brain blood circulation during a time period of at least 10 minutes, for example during a time period of at least 30 minutes, at least 1 hours, at least 12 hours, or any intermediate, shorter or longer time period.

According to some exemplary embodiments, during the monitoring the system collects information comprising measurement data and/or output from one or more models, for example models that detect hemodynamic changes. In some embodiments, the system stores the information in a memory associated with the system, for example a memory in the control unit and/or a memory of a remote device, for example a remote server or a remote computer that is in communication with a sensing unit and/or a control unit of the system. In some embodiments, the remote device processes the information, for example in order to generate a database, to classify the information, and/or to generate improved models.

According to some exemplary embodiments, the system is configured to deliver an alert signal to a subject if the system identifies hemodynamic changes that are not within a normal range of values, and/or if the system identifies hemodynamic changes that indicate an ischemic event, for example a stroke in the subject. Alternatively or additionally, the system transmits the alert signal to a remote device and/or transmits the alert signal to emergency services, for example in order to provide immediate help and support to the subject. In some embodiments, the alert delivered to the remote device and/or to the emergency services comprises at least one of, medical information, medical history, measurements acquired from the subject, time related parameters, for example duration of an ischemic event, initiation time of the ischemic event and/or of the abnormal hemodynamic changes

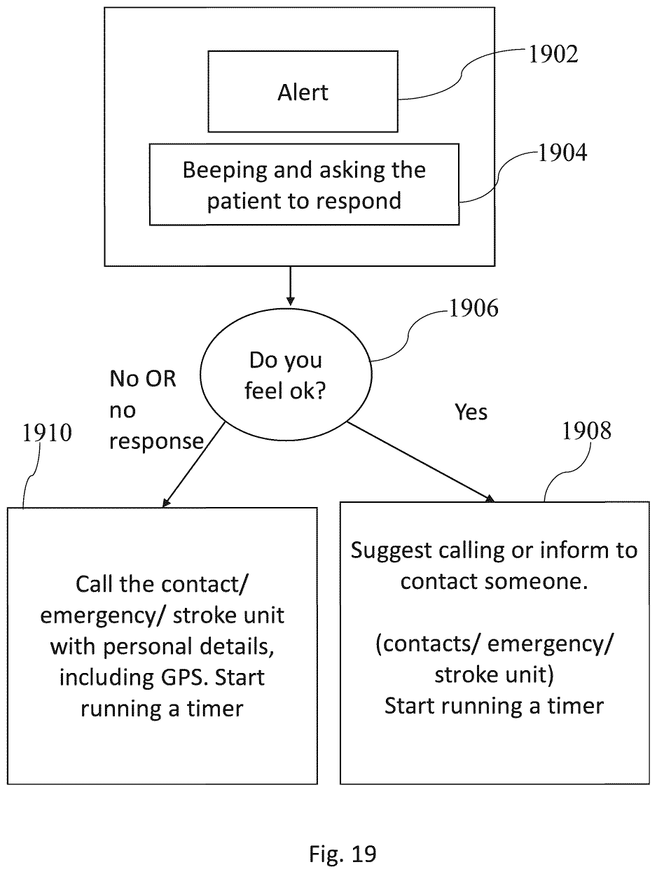

Reference is now made to FIG. 19, depicting a process for managing a subject, for example a patient, when receiving an alert indication, according to some exemplary embodiments of the invention.

According to some exemplary embodiments, an alert indication is generated by the system, at block 1902. Optionally, the system generates an alert signal which includes an audio signal, for example beeping with a request to communicate with the subject, for example asking the patient to respond, at block 1904. In some embodiments, the system communicates with the patient directly or via a remote device of the patient, for example a mobile device, a cellular device, a virtual assistant device, and/or a wearable device coupled to the patient.

According to some exemplary embodiments, the system receives input regarding the state of the patient, for example from one or more cameras, microphones, and/or sensors of the system or of any other device in communication with the system that is located at the vicinity of the patient. In some embodiments, the system receives input regarding the state of the patient by asking the patient questions, at block 1906, for example “do you feel ok?”.

According to some exemplary embodiments, if the patient does not respond, the system communicates at block 1910 with at least one of, a caregiver, an emergency center, an emergency service, a telemedicine service, and/or a physician. The system optionally provides information regarding the state of the patient, medical history, measurements acquired form the patient and/or time parameters related to the initiation of the stroke event and/or duration of the stroke event.

According to some exemplary embodiments, if the patient responds, the system suggests the patient to call at least one of, a caregiver, an emergency center, an emergency service, a telemedicine service, and/or a physician.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method for determining changes in brain blood circulation, comprising:
recording signals from a single side of a neck;
processing the recorded signals;
determining hemodynamic changes in brain blood circulation indicating abnormal blood circulation at a contralateral side, based on results of said processing.

2. A method according to claim 1, wherein said determined hemodynamic changes indicate a pending ischemic event in the brain.

3. A method according to claim 2, comprising one or more of:
a. delivering an alert signal if said determined hemodynamic changes indicate said pending ischemic event; wherein said alert signal includes information regarding initiation of a therapeutic time window and/or a duration of the therapeutic window; wherein said delivering comprises delivering said alert signal to said subject and/or to a remote device;
b. detecting a location of said abnormal blood circulation and/or a location of a stenosis in a blood vessel, based on said determined hemodynamic changes, and wherein said delivered alert comprises information regarding said detected location.

4-6. (canceled)

7. A method according to claim 1, comprising attaching at least one sensing unit to a skin surface of said neck at said single side, and wherein said recording comprises recording said signals by at least one detector of said at least one sensing unit; and
wherein said attaching comprises attaching said at least one sensing unit to a skin surface above a carotid artery in the neck.

8. (canceled)

9. A method according to claim 1, comprising implanting at least one detector at least partly into the neck or under the skin surface, at said single side, and wherein said recording comprise recording said signals by said at least one detector; and
wherein said implanting comprises one or more of:
a. implanting said at least one detector near a carotid artery;
b. subcutaneously implanting said at least one detector.

10-11. (canceled)

12. A method according to claim 1, comprising one or more of:
a. measuring and at least one pulse wave based on said recorded signals, and wherein said identifying comprises identifying said hemodynamic changes based on said pulse wave measurements;
b. measuring electrocardiogram (ECG), and wherein said identifying comprises identifying said hemodynamic changes based on said ECG and pulse wave measurements;
c. recording acoustic signals, and wherein said identifying comprises identifying said hemodynamic changes based on said acoustic signals and said ECG and pulse wave measurements;
wherein said processing comprises extracting features which comprise morphological features and/or time based features from said at least one measured pulse wave, and wherein said determining comprises determining said hemodynamic changes based on said extracted features; and wherein said processing comprises determining quality of said extracted features and/or in said at least one pulse wave, prior to said determining.

13-16. (canceled)

17. A method according to claim 1, wherein said determining hemodynamic changes in brain blood circulation comprises determining hemodynamic changes in blood vessels delivering blood to the brain and/or blood vessels surrounding the brain, at said contralateral side.

18-38. (canceled)

39. A system for determining changes in brain blood circulation, comprising:
at least one sensing unit configured to be positioned at a side of a neck, comprising at least one pulse wave detector for measuring pulse waves;
a control unit functionally coupled to said at least one sensing unit, comprising:
a memory circuitry;
a control circuitry configured to receive said pulse wave measurement, to process said pulse wave measurements, and to determine hemodynamic changes in brain blood circulation indicating abnormal blood circulation at a contralateral side based on said processing results.

40. A system according to claim 39, wherein said control circuitry is configured to determine changes in said brain blood circulation indicating abnormal blood circulation at an ipsilateral side, based on said processing results.

41. A system according to claim 39, wherein said at least one sensing unit comprises one or more of:
a. at least one detector for measuring electrocardiogram (ECG), and wherein said control circuitry is configured to receive said ECG measurements and to process said ECG and pulse wave measurement;
b. at least one acoustic sensor configured to measure sound waves, and wherein said control circuitry is configured to receive said measured sound waves, and to determine said changes in said brain blood circulation based on said measured sound waves.

42. (canceled)

43. A system according to claim 39, wherein said at least one pulse wave detector comprises at least two waves detectors for measuring at least two pulse waves.

44. A system according to claim 39, wherein said at least one sensing unit is one or more of:
a. an implantable sensing unit comprising a flexible casing having an outer flat and smooth surface, wherein said flexible casing is shaped and sized to implanted into neck tissue;
b. a flexible skin patch configured to be attached to the skin surface via an adhesive layer on at least one surface of said skin patch.

45. (canceled)

46. A system according to claim 39, wherein said control circuitry is configured to perform one or more of:
a. process said pulse waves measurements by extracting morphological features and/or time based features from said pulse waves measurements, and to determine said changes in brain blood circulation based on said extracted morphological features and/or said extracted time based features; wherein said morphological features comprise at least one of, waveform amplitude, frequency, number of peaks, area under a curve, width, angle of slopes, timing, amplitude of peaks and/or shape;
b. provide said extracted morphological features and/or said extracted time based features as input data to a stroke detecting model stored in said memory circuitry, and to determine changes in brain blood circulation indicating said abnormal blood circulation which indicates a pending ischemic event, based on an output of said stroke detecting model;
c. provide said pulse waves measurements as input data to said stroke detecting model;
d. determine quality of said pulse waves measurements, said extracted morphological features and/or said extracted time based features, prior to using said stroke detecting model.

47. (canceled)

48. A system according to claim 46, wherein said time based features comprise time intervals between specific points in a waveform signal.

49-51. (canceled)

52. A system according to claim 46, wherein said control unit comprises one or more of:
a. a user interface configured to generate a human detectable indication, and wherein said control circuitry signals said user interface to generate said human detectable indication when changes in brain blood circulation indicating abnormal blood circulation are determined and/or when changes in brain blood circulation indicating a pending ischemic event are determined;
b. a communication circuitry, and wherein said communication circuitry is configured to deliver an alert signal to a remote device when said changes in brain blood circulation are determined.

53. (canceled)

54. A device for detecting an ischemic event in a subject, comprising:
a memory circuitry, wherein said memory circuitry stores pulse waves measurements;
a control circuitry, wherein said control circuitry is configured to extract features comprising morphological features and/or time based features, from said pulse waves measurements, and to identify changes in brain blood circulation indicating a pending ischemic event based on said extracted features.

55. A device according to claim 54, wherein said memory circuitry stores electrocardiogram (ECG) measurements, and wherein said control circuitry is configured to identify changes in said brain blood circulation indicating stroke pending ischemic event based on said extracted features and said ECG measurements;
wherein said control circuitry is configured to select at least one set of pulse waves measurements from said stored pulse waves measurements using said ECG measurements, and to extract said features from said at least one selected set.

56. (canceled)

57. A device according to claim 54, wherein said morphological features comprise at least one of, waveform amplitude, frequency, number of peaks, area under a curve, width, angle of slopes, timing, amplitude of peaks and/or shape; and wherein said time based features comprise time intervals between specific points in a waveform signal.

58. (canceled)

59. A device according to claim 54, wherein said memory stores one or more of:
a. a stroke detecting model, and wherein said control circuitry is configured to insert said extracted features and/or said pulse waves measurements as input data to said stroke detecting model, and to identify changes in brain blood circulation indicating stroke pending ischemic event based on an output of said stroke detecting model;
b. acoustic signals, and wherein said control circuitry is configured to perform one or more of:
i. insert said acoustic signals as input data to said stroke detecting model;
ii. determine quality of said extracted features and/or quality of said stored pulse waves measurements prior to providing input data to said stroke detecting model.

60-61. (canceled)

62. A device according to claim 54, comprising one or more:
a. a communication circuitry, and wherein said communication circuitry signals said communication circuitry to deliver signals to a remote device with information regarding a detected pending ischemic event according to said identified changes in brain blood circulation indicating said pending ischemic event;
b. a user interface configured to generate a human detectable indication, and wherein said control circuitry signals said user interface to generate said human detectable indication with information regarding a detected pending ischemic event according to said identified changes in brain blood circulation indicating said pending ischemic event.

63. (canceled)
